(19) United States
(12) Patent Application Publication
(10) Pub. No.: US 2010/0215684 A1
(43) Pub. Date: Aug. 26, 2010

(54) STREPTOCOCCUS M PROTEIN, IMMUNOGENIC FRAGMENTS, NUCLEIC ACIDS AND METHODS OF USE

(75) Inventors: Andrew Cartner Barnes, St Lucia Queensland (AU); Justice Craig Forrester Baiano, Eaton Hills Queensland (AU)

Correspondence Address: KNOBBE MARTENS OLSON & BEAR LLP, 2040 MAIN STREET, FOURTEENTH FLOOR, IRVINE, CA, 92614, US

(73) Assignee: THE UNIVERSITY OF QUEENSLAND, ST. LUCIA QSLD (AU)

(21) Appl. No.: 12/739,399

(22) PCT Filed: Oct. 24, 2008
(86) PCT No.: PCT/AU2008/001570

§ 371 Date: Apr. 22, 2010

Related U.S. Application Data
(60) Provisional application No. 60/982,540, filed on Oct. 25, 2007.

(51) Int. Cl. A61K 39/02; C07K 14/00; C07K 7/08; C07K 16/12; C07H 21/00; C12N 15/74; C12N 5/10; A61K 38/16; A61K 38/10; G01N 33/53; A61P 37/04; A61P 31/04

(52) U.S. Cl. 424/190.1; 530/350; 530/326; 530/387.9; 536/23.7; 435/320.1; 435/325; 435/252.3; 514/13; 514/12; 435/7.1

(57) ABSTRACT

Isolated M proteins, or M-like proteins, of Streptococcus iniae, encoding nucleic acids, genetic constructs and antibodies which bind the isolated M proteins are provided. Also provided are isoforms and/or fragments of the M proteins, or M-like proteins, that display reduced fibrinogen binding. The isolated M proteins, antibodies and encoding nucleic acids maybe useful for diagnosis, immunization and/or therapy of Streptococcus iniae infections of animals such as fish and humans.

### FIELD OF THE INVENTION

THIS INVENTION relates to particular proteins and nucleic acids. More particularly, this invention relates to isolated proteins and nucleic acids of Streptococcus iniae and their use in diagnosis and/or therapy of Streptococcus iniae infections in animals, particularly fish.

### BACKGROUND OF THE INVENTION

Streptococcus iniae has become one the most serious aquatic pathogens in the last decade causing high losses in farmed marine and freshwater finfish in warmer regions. First identified in 1976 from a captive Amazon freshwater dolphin, from which the bacterium derives its name. S. iniae is globally distributed throughout warm water finfish aquaculture. A review of the fish species so far identified as susceptible to S. iniae infection is provided in Agnew & Barnes, 2007, Vet. Microbiol. 122 1.

S. iniae also has zoonotic potential, with human infections identified in the USA, Canada, and throughout Asia. In humans, infection is clearly opportunistic with all cases to date associated with direct infection of puncture wounds during preparation of contaminated fish, and generally in elderly or immunocompromised individuals (Agnew & Barnes, 2007, supra).

Antibiotics have been shown to be effective in treating some fish infections. Enrofloxacin, oxytetracycline, furazolidone and amoxicillin have been found to be useful for treating S. iniae infections in fish, although the efficay of each varies from species to species. However, the use of antimicrobials in aquaculture situations does have some limitations and concerns. The first is selection for resistance amongst dense populations. The second is that drug residues are of concern in farmed fish destined for human consumption.

A more recently developed approach to S. iniae control is through vaccination. A program was successfully initiated in farmed rainbow trout in Israel from 1995 to 1997 using autogenous vaccines consisting of whole-cell formalin inactivated S. iniae injected intra-peritoneally (Bercovier et al., 1996, Immunization with Bacterial Antigens: infections with streptococci and related organisms. Second International Symposium in Fish Vaccinology pp 153-60; Eldar et al., 1997, Vet. Immunol. Immunpathol. 56 175). Fish were protected for over 4 months, covering the majority of the short trout production cycle in Israel (Bercovier et al., 1996, supra). Large-scale vaccination programs in Upper Galilee reduced mortalities due to S. iniae from 50% to less than 5% annually (Eldar et al., 1997, supra). Evidence suggested that the basic mechanism of protection was antibody mediated, probably generated in response to heat stable, protein based antigenic determinants (Bercovier et al., 1996, supra).

Evidence for the role of antibody in conferring protection is supported by data showing that passive immunization of tilapia with anti-S. iniae sera was also protective (Shelby et al., 2002, J. Fish. Dis. 25 1). However, the success of the vaccination program was short-lived. In 1997 massive new outbreaks occurred due to a new variant of the bacterium. Unlike the previous isolates, this variant was arginine dihydrolase and ribose negative, and seemed to have shifted its capsular composition (Bachrach et al., 2001, Appl. Environ. Microbiol. 67 3756; Zlotkin et al., 1998, Appl. Environ. Microbiol. 64 4065). The vaccination program in Israel was shown to have allowed some pathogen to remain in fish or the environment and provided enough selective pressure for a distinctly different serotype to become dominant (Bachrach et al., 2001, supra).

More recently, two new vaccines have become available in parts of Asia to protect against S. iniae infection. One is a monovalent inactivated vaccine (Norvax1 Strep Si) that can be used as an immersion or an injectable. Schering-Plough has developed AquaVac™ Garvetil™, which combines protection against S. iniae and Lactococcus garvieae, and can be given either as an immersion or orally in feed.

Despite the existence of S. iniae vaccines, this pathogen is still a major problem in fish and other animals. Therefore need exists to identify and isolate molecular components of S. iniae that are useful in diagnosis and/or therapy of S. iniae infection in animals.

### SUMMARY OF THE INVENTION

The present invention is directed to an isolated M protein, or M-like protein, of Streptococcus iniae which, in particular non-limiting forms, may exhibit relatively reduced fibrinogen binding, and isolated nucleic acids encoding said M protein, or M-like protein.

The present invention is also directed to compositions and methods for treatment and/or diagnosis of S. iniae infections in animals.

In one aspect, the invention provides an isolated M protein, or M-like protein, of S. iniae.

In one embodiment, the isolated M protein, or M-like protein, may comprise an amino acid sequence selected from the group consisting of: RLTLEEKMEALRKVVT (SEQ ID NO:1) and KMAEIQEEANKKIAA (SEQ ID NO:2).

In particular embodiments, the isolated M protein, or M-like protein, of S. iniae may comprise an amino acid sequence according to SEQ ID NO:3 or SEQ ID NO:4.

In another aspect, the invention provides an isolated protein having reduced or lower fibrinogen binding compared to an isolated protein comprising an amino acid sequence according to SEQ ID NO:3 or SEQ ID NO:4, said isolated protein comprising an amino acid sequence of an S. iniae M protein, or M-like protein, but which does not comprise an amino acid sequence selected from the group consisting of: RLTLEEKMEALRKVVT (SEQ ID NO:1); KMAEIQEEANKKIAA (SEQ ID NO:2); and one or more of residues 190-220 of SEQ ID NO:3 or SEQ ID NO:4.

The isolated protein having reduced fibrinogen binding may comprise an amino acid sequence selected from the group consisting of: SEQ ID NO:5; SEQ ID NO:6 and SEQ ID NO:7.

In another aspect, the invention provides an isolated protein comprising an amino acid sequence selected from the group consisting of: SEQ ID NO:1; SEQ ID NO:2; SEQ ID NO:3; SEQ ID NO:4; SEQ ID NO:5; SEQ ID NO:6; and SEQ ID NO:7.

In another aspect, the invention provides a fragment, variant or derivative of an isolated protein according to any of the aforementioned aspects.

In another aspect, the invention provides an isolated nucleic acid encoding an isolated protein of, or a fragment, variant or derivative thereof, according to the aforementioned aspects.

The isolated nucleic acid may comprise a nucleotide sequence set forth in any one of SEQ ID NOS: 8-11 and 39.

In another aspect, the invention provides a genetic construct comprising an isolated nucleic acid according to any of the aforementioned aspects.

In another aspect, the invention provides a host cell comprising a genetic construct according to the aforementioned aspect.

In another aspect, the invention provides an antibody which binds an isolated M protein, or M-like protein, of S. iniae, fragment, variant or derivative thereof.

In another aspect, the invention provides a composition comprising an isolated M protein, or M-like protein, of S. iniae, fragment, variant or derivative thereof, or an antibody thereto, according to the aforementioned aspects together with a suitable carrier diluent or excipient.

The composition may be an immunotherapeutic composition capable of eliciting an immune response against S. iniae.

The composition may be a vaccine capable of eliciting a protective immune response against S. iniae.

In another aspect, the invention provides a method of treating an S. iniae infection of an animal, said method including the step of administering a composition comprising an isolated M protein, or M-like protein, of S. iniae, fragment, variant or derivative thereof, or an antibody thereto, together with a suitable carrier diluent or excipient, to an animal infected with S. iniae to thereby treat said infection.

In another aspect, the invention provides a method of immunizing an animal against S. iniae, said method including the step of administering to an animal a composition comprising an isolated M protein, or M-like protein, of S. iniae, fragment, variant or derivative thereof, or an antibody thereto, together with a suitable carrier diluent or excipient, to thereby immunize the animal.

In another aspect, the invention provides a method of determining whether an animal is, or has been, exposed to S. iniae bacteria or components thereof, said method including the step of determining whether a biological sample obtained from said animal comprises an isolated protein or fragment, variant or derivative thereof, wherein the presence of said isolated protein or fragment, variant or derivative thereof, indicates that said animal is, or has been, exposed to S. iniae bacteria or molecular components thereof.

In another aspect, the invention provides a method of determining whether an animal is, or has been, exposed to S. iniae bacteria or components thereof, said method including the step of determining whether a biological sample obtained from said animal comprises an isolated nucleic acid according to any of the aforementioned aspects, wherein the presence of said isolated nucleic acid agment indicates that said animal is, or has been, exposed to S. iniae bacteria or molecular components thereof.

In another aspect, the invention provides a method of determining whether an animal is, or has been, exposed to S. iniae bacteria or components thereof, said method including the step of determining whether a biological sample obtained from said animal comprises an antibody or antibody fragment that binds an isolated M protein, or M-like protein, of S. iniae, wherein the presence of said antibody or antibody fragment indicates that said animal is, or has been, exposed to S. iniae bacteria or molecular components thereof.

In another aspect the invention provides a diagnostic kit and/or diagnostic composition comprising one or more detection agents for detecting S. iniae bacteria, molecular components thereof or an antibody thereto.

Diagnostic kits and/or compositions may comprise one or more detection agents suitable for nucleic acid-based or protein-based detection and comprise one or more antibodies, probes and/or primers to facilitate detection of an S. iniae M protein, or M-like protein, encoding nucleic acids and/or fragments thereof.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1 S. iniae strain QMA72 M protein-encoding nucleotide sequence (SEQ ID NO:8).

FIG. 2. S. iniae strain QMA76 M protein-encoding nucleotide sequence (SEQ ID NO:9).

FIG. 3. S. iniae strain QMA141 M protein-encoding nucleotide sequence (SEQ ID NO:10).

FIG. 4A. S. iniae strain QMA136 M protein-encoding nucleotide sequence (SEQ ID NO:11). The nucleotide sequence comprises separate open reading frames (residues 1-609 and residues 677-1606) that respectively encode a QMA136 M protein N-terminal amino acid sequence (SEQ ID NO:6) and a QMA136 M protein C-terminal amino acid sequence (SEQ ID NO:7).

FIG. 4B. S. iniae strain QMA136 M protein N-terminal amino acid sequence (SEQ ID NO:6)

FIG. 4C. S. iniae strain QMA136 M protein C-terminal amino acid sequence (SEQ ID NO:7).

FIG. 5. Amino acid alignments of S. iniae M protein amino acid sequences SEQ ID NOS:3-7).

FIG. 6. Amino acid sequence alignment of M proteins from isolates QMA0072 (SEQ ID NO:3), QMA0076 (SEQ ID NO:4) and QMA0141 (SEQ ID NO:5) with S. dysgalactiae demA gene product (CAB65411; SEQ ID NO:12).

FIG. 7. Nucleotide and amino acid sequences of S. iniae simA gene (SEQ ID NO:39) and M protein (SEQ ID NO:4). Putative Mgx protein binding site is bolded, putative promoter sequences (−10 and −35 boxes and ribosome binding site—RBS—sequence) are boxed, inverted repeats are highlighted by wedges, the membrane anchor is italicised, the stop codon is bolded and indicated by an asterisk.

FIG. 8. Expression and western blotting of M proteins. Panel A: Coomassie blue-stained proteins from E. coli expression lysates. Lane 1—molecular weight marker, lane 2—control lysate, lane 3—QMA0072, lane 4—QMA0076, lane 5—QMA0141. Panel B: Western blot detection of M proteins with biotinylated fibrinogen. Lanes 1-5 correspond to lanes in Coomassie blue-stained gel.

FIG. 9. Effect of fibrinogen binding on activation of barramundi macrophages by S. iniae. S. iniae isolate QMA0072 harvested in exponential phase was incubated with fibrinogen, BSA or HBSS and the respiratory burst was measured by luminol enhanced chemiluminescence.

FIG. 10. Kaplan-Meyer survival curves for S. iniae vaccine trial.

FIG. 11. Relative percent survival for two vaccines based on 2 replicates (BL21 simA) or a single trial (pUK21 simA). RPS was calculated with respect to appropriate controls (E. coli BL21 or empty pUK21 vector).

### DETAILED DESCRIPTION OF THE PREFERRED EMBODIMENT

The present invention relates to the isolation and characterization of S. iniae M protein isoforms which may be useful in diagnosis and/or treatment of S. iniae infection of animals, including but not limited to fish and mammals such as humans and dolphins.

In one aspect, the invention provides an isolated M protein, or M-like protein, of S. iniae.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein, an “M protein of S. iniae” or an “M-like protein of S. iniae” is a protein that is obtainable from an S. iniae bacterium and/or comprises an amino acid sequence of a protein that is obtainable from an S. iniae bacterium, which protein is structurally and functionally orthologous, or at least related in the case of an M-like protein, to an M protein of another Group A streptococcus.

An “M protein” is a protein of a Group A streptococcus that is typically found on a bacterial cell wall extending into the surrounding capsule. The M protein may be variable among different serotypes. The M protein may also confer streptococcal virulence by protecting the bacterial cell from phagocytic action.

By “protein” is meant an amino acid polymer. The amino acids may be natural or non-natural amino acids, D- or L-amino acids as are well understood in the art.

A “peptide” is a protein having less than fifty (50) amino acids.

A “polypeptide” is a protein having fifty (50) or more amino acids.

Various aspects of the invention provide an isolated M protein of S. iniae, including fragments, variants and derivatives of said M protein.

In particular aspects, said isolated M protein of S. iniae comprises an amino acid sequence set forth in SEQ ID NO:3 or SEQ ID NO:4.

The invention also provides S. iniae M protein isoforms, putatively arising from a 40 nucleotide insertion in an M protein gene, that do not comprise at least one of the amino acid sequences RLTLEEKMEALRKVVT (SEQ ID NO:1) and KMAEIQEEANKKIAA (SEQ ID NO:2).

Examples of these proteins are provided herein as SEQ ID NOS: 6 and 7. Data provided hereinafter show that these M proteins display reduced fibrinogen binding.

Furthermore, an M protein comprising an amino acid sequence set forth in SEQ ID NO:5 has neither of the amino acid sequences RLTLEEKMEALRKVVT (SEQ ID NO:1) and KMAEIQEEANKKIAA (SEQ ID NO:2) and displays reduced fibrinogen binding

In addition, or alternatively, M proteins that lack or one or more of residues 190-220 of SEQ ID NO:3 or SEQ ID NO:4 may bind reduced or lower levels of fibrinogen.

While the absence of RLTLEEKMEALRKVVT (SEQ ID NO:1), KMAEIQEEANKKIAA (SEQ ID NO:2) and/or one or more of residues 190-220 of SEQ ID NO:3 or SEQ ID NO:4 is a feature of M proteins, or M-like proteins, that bind reduced or lower levels of fibrinogen, it is not necessarily the case that any of these amino acid sequences constitute fibrinogen binding sites or contribute to fibrinogen binding.

Suitably, fibrinogen binding is reduced compared to an isolated M protein comprising an amino acid sequence selected from the group consisting of: SEQ ID NO:3 and SEQ ID NO:4.

By “reduced or lower” in this context is meant less than 99%, in one aspect less than 95%, in one aspect less than 90%, in one aspect less than 75%, and in another aspect, less than 50% or less than 40,%, 30%, 25%, 20%, 15%, 10%, 5%, 3%, 2% or 1% of the amount of fibrinogen bound by an M protein having amino acid sequences according to SEQ ID NO:3 or SEQ ID NO: 4, “molecule for molecule”.

Without wishing to be bound by any particular theory, it is proposed that fibrinogen may mask immune responses to S. iniae M protein, in which case isolated proteins having reduced fibrinogen binding may be particularly useful immunogens for administration to animals.

As used herein. “animals” include and encompass any animal susceptible to S. iniae infection, including but not limited to fish and mammals such as humans and dolphins.

The term “fish” as used herein includes within its scope: Agnatha, jawless fish such as the hagfish and lampreys; Chrondrichthyes, fish whose skeleton is made of cartilage; and Osteichthyes, fish whose skeleton is composed mostly of bone. Osteichthyes comprise two main groups: ray-finned fish and the lobe-finned fish. In one aspect of the present invention, the fish is a ray-finned fish.

Non-limiting examples of fish susceptible to S. inae infection to which the present invention may apply include commercially important fish such as salmon, barramundi, seabass, flounder, trout, bream, snapper, Nile perch, tilapia, mullet, cod and yellowtail.

A more extensive list of fish susceptible to S. inae infection is provided in Agnew & Barnes, 2007, supra.

In another aspect, the invention provides a fragment, variant or derivative of an isolated M protein of S. iniae inclusive of isolated proteins having reduced fibrinogen binding.

A “protein fragment” is a segment, domain, portion or region of a protein, which constitutes less than 100% of the amino acid sequence of the protein.

For example, protein fragments may comprise up to 99%, 95%, 90%, 85%, 80%, 75%, 70%, 65%, 60%, 55%, 50%, 45%, 40%, 35%, 30%, 25%, 20%, 15%, 10%, 5%, 3%, 2% or 1% of said protein.

In particular aspects, a protein fragment may comprise, for example, at least 5, 10, 20, 30, 40, 50 60, 70, 80, 90, 100, 120, 140, 150, 200, 250, 300, 350, 400, 450 or 500 contiguous amino acids of an S. iniae M protein.

A peptide may be a protein fragment, for example comprising at least 6, 10, 12, 15, 20, 30, 40 and up to 50 contiguous amino acids.

Protein fragments, inclusive of peptide fragments, may be obtained through the application of standard recombinant nucleic acid techniques or synthesized using conventional liquid or solid phase synthesis techniques. For example, reference may be made to solution synthesis or solid phase synthesis as described, for example, in Chapter 18 of CURRENT PROTOCOLS IN PROTEIN SCIENCE, Coligan et al. Eds (John Wiley & Sons, 1995-2000). Alternatively, peptides can be produced by digestion of a polypeptide of the invention with proteinases such as endoLys-C, endoArg-C, endoGlu-C and V8-protease. The digested fragments can be purified by chromatographic techniques as are well known in the art.

In various aspects of the invention, the protein fragment may be a “biologically active protein fragment” which displays at least 25%, more preferably at least 50% and even more preferably at least 70%, 75%, 80%, 85%, 90%, 95% or up to 100% of the biological activity of a full length S. iniae M protein. Biological activity may be in terms of immunogenicity, antigenicity and/or fibrinogen binding, although without limitation thereto.

Non-limiting examples of such protein fragments include isolated M protein fragments that do not comprise or lack one or the other of the amino acid sequences RLTLEEKMEALRKVVT (SEQ ID NO:1) and KMAEIQEEANKKIAA (SEQ ID NO:2).

Specific examples of such fragments comprise amino acid sequences set forth in SEQ ID NOS: 6 and 7, respectively, which display relatively reduced fibrinogen binding.

Alternatively, the biologically active protein fragment may display enhanced activity compared to a full length S. iniae M protein.

Protein fragments comprising amino acid sequences set forth in SEQ ID NOS: 6 and 7, respectively, may display increased or enhanced immunogenicity compared to a full length S. iniae M protein.

In other embodiments, biologically-active fragments may include mature, processed forms of the M proteins of the invention.

For example, biologically-active fragments may lack N-terminal signal sequences. Non-limiting examples include N-terminally processed M proteins lacking amino acids 1-41.

The present invention also provides variants of the isolated proteins of the invention.

“Variants” include within their scope naturally-occurring variants such as allelic variants, orthologs and homologs and artificially created mutants, for example.

The terms “mutant”, “mutation” and “mutated” are used herein generally to encompass conservative or non-conservative amino acid substitutions, deletions and/or insertions introduced into an isolated protein or fragment thereof.

Generally, protein variants have at least 80% amino acid sequence identity to an isolated protein of the invention such as set forth in any one of SEQ ID NOS:1-7. In certain aspects of the present invention, protein variants have at least 85%, 90%, 91%, 92%, 93%, 94%, 95%, 96%, 97%, 98% or 99% amino acid sequence identity to an isolated protein of the invention such as set forth in any one of SEQ ID NOS:1-7.

Terms used herein to describe sequence relationships between respective nucleic acids or proteins include “comparison window”, “sequence identity”, “percentage of sequence identity” and “substantial identity”. Because respective nucleotide or amino acid sequences may each comprise: (1) only one or more portions of a complete sequence that are shared by respective nucleic acids or proteins, and (2) one or more portions which are divergent between the nucleic acids or proteins, sequence comparisons are typically performed by comparing sequences over a “comparison window” to identify and compare local regions of sequence similarity. A “comparison window” refers to a conceptual segment of typically at least 6, 10, 12, 20 or more contiguous residues that is compared to a reference sequence. The comparison window may comprise additions or deletions (i.e., gaps) of about 20% or less (e.g. 5, 10 or 15%) as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

As used herein, a “derivative protein” is a protein which comprises one or more chemical and/or structural modifications or alterations of an amino acid sequence of a naturally-occurring M protein of S. iniae.

By way of example only, derivative proteins may comprise one or more modifications inclusive of amino acid side chain modifications, non-natural amino acids, glycosylated amino acid residues, cross-linked amino acid residues and/or additional amino acid residues.

Examples of amino acid side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Derivatives also include within their scope isolated fusion proteins comprising additional amino acid sequences such as N- or C-terminal fusion partner sequences. Fusion partners assist identification and/or purification of a fusion protein comprising said fusion partner.

Well known examples of fusion partners include, but are not limited to, glutathione-S-transferase (GST), Fc and hinge portion of human IgG, maltose binding protein (MBP) and hexahistidine (HIS₆), which are particularly useful for isolation of the fusion protein by affinity chromatography. For the purposes of fusion protein purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system.

In some cases, fusion partners also have protease cleavage sites, such as for Factor Xₐ or Thrombin, which allow the relevant protease to partially digest the fusion protein described herein and thereby liberate the protein of the invention therefrom. The liberated protein can then be isolated from the fusion partner by subsequent chromatographic separation.

Fusion partners also include within their scope “epitope tags”, which are usually short peptide sequences for which a specific antibody is available. Well-known examples of epitope tags for which specific monoclonal antibodies are readily available include c-myc, influenza virus haemagglutinin and FLAG tags.

Isolated S. iniae M proteins of the invention, inclusive of fragments, variants and derivatives, may be made in recombinant or chemical synthetic form.

Generally, recombinant proteins may be conveniently prepared by a person skilled in the art using standard protocols commonly known in the art.

Chemical synthesis is optimally utilized for peptides and other protein fragments that do not exceed 60-80 contguous amino acids in length. Such methods are well known in the art.

M proteins of S. iniae may be inducibly expressed and at least partly refractory to expression under standard laboratory media under normal culture conditions. Expression of M proteins may be therefore facilitated by inclusion of inducing agents during bacterial cell culture. Non-limiting examples of metals, chelators, serum proteins and/or other additives that induce or maximise bacterial expression of S. iniae M proteins.

One aspect of the invention also includes antibodies which bind, recognize and/or have been raised against isolated proteins of the invention, fragments, variants or derivatives thereof. Antibodies also include antibody fragments such as Fc fragments, Fab and Fab′2 fragments, diabodies, Fv and scFv fragments. Antibodies may be monoclonal or polyclonal. Antibodies may be made in suitable production animal such as a mouse, rat, rabbit, sheep, chicken or goat.

Alternatively, antibodies may be isolated from fish or other animals that have been naturally exposed to S. iniae or an M protein thereof.

Monoclonal antibodies may be produced by standard methods such as described in CURRENT PROTOCOLS IN IMMUNOLOGY (Eds. Coligan et al. John Wiley & Sons. 1995-2000) and Harlow, E. & Lane, D. Antibodies: A Laboratory Manual (Cold Spring Harbour, Cold Spring Harbour Laboratory, 1988). Such methods generally involve obtaining antibody-producing cells, such as spleen cells, from an animal immunized as described above, and immortalizing said cell, such as by fusion with an immortalized fusion partner cell.

Monoclonal antibodies, or antigen-binding fragments of same may also be produced by recombinant means. Such recombinant methods are well known in the art and a variety of commercial sources are available for production of recombinant antibodies.

As is well understood in the art, antibodies may be conjugated with labels selected from a group including an enzyme, a fluorophore, a chemiluminescent molecule, biotin, radioisotope or other label.

Examples of suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution or with a suitable chromogenic or chemiluminescent substrate.

Examples of chromogens include diaminobanzidine (DAB), permanent red, 3-ethylbenzthiazo line sulfonic acid (ABTS), 5-bromo-4-chloro-3-indolyl phosphate (BCIP), nitro blue tetrazolium (NBT), 3,3′,5,5′-tetramethyl benzidine (TNB) and 4-chloro-1-naphthol (4-CN), although without limitation thereto.

A non-limiting example of a chemiluminescent substrate is Luminol™, which is oxidized in the presence of horseradish peroxidase and hydrogen peroxide to form an excited state product (3-aminophthalate).

Fluorophores may be fluorescein isothiocyanate (FITC), tetramethylrhodamine isothiocyanate (TRITC), allophycocyanin (APC), Texas Red (TR), Cy5 or R-Phycoerythrin (RPE), although without limitation thereto.

Radioisotope labels may include ¹²⁵I, ¹³¹I, ⁵¹Cr and ⁹⁹Tc, although without limitation thereto.

Other antibody labels that may be useful include colloidal gold particles and digoxigenin.

The invention also provides isolated nucleic acids that encode the isolated M proteins described herein, inclusive of fragments, variants and derivaties of said isolated M proteins.

In particular aspects, the invention provides an isolated nucleic acid comprising a nucleotide sequence set forth in any one of SEQ ID NOS: 8-11 and 39. With particular regard to SEQ ID NO:11, the nucleotide sequence comprises separate open reading frames defined by residues 1-609 and residues 677-1606.

The term “nucleic acid” as used herein designates single- or double-stranded mRNA, RNA, cRNA, RNAi, siRNA and DNA inclusive of cDNA, mitochondrial DNA (mtDNA) and genomic DNA.

A “polynucleotide” is a nucleic acid having eighty (80) or more contiguous nucleotides, while an “oligonucleotide” has less than eighty (80) contiguous nucleotides.

Non-limiting examples of oligonucleotides are provided in Tables 1 and 3 (SEQ ID NOS:18-38).

The invention also provides nucleic acid variants including homologous, orthologous and mutant nucleotide sequences.

In one aspect, the invention provides a variant nucleic acid having at least 70%, 75%, 80%, 85%, 90%, 91%, 92%, 93%, 94%, 95%, 96%, 96%, 98% or 99% nucleotide sequence identity to an isolated nucleic acid of the invention.

A variant nucleic acid of the invention may hybridize an isolated nucleic acid of the invention under high stringency conditions.

Stringency conditions are well known in the art, such as described in Chapters 2.9 and 2.10 of Ausubel et al. CURRENT PROTOCOLS IN MOLECULAR BIOLOGY (John Wiley & Sons NY, 1995-2006) and in particular at pages 2.9.1 through 2.9.20.

Generally, stringency may be varied according to the concentraction of one or more factors during hybridization and/or washing. Such factors may include ionic strength, detergent type and/or concentration, temperature, denaturant type and/or concentration, as are well understood in the art.

Specific, non-limiting examples of high stringency conditions appropriate for obtaining isolated nucleic acids of the invention include:—

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

In general, washing is carried out at Tₘ=69.3+0.41 (G+C) %−12° C. In general, the Tₘ of a duplex DNA decreases by about 1° C. with every increase of 1% in the number of mismatched bases.

It will therefore be appreciated that specific examples of variants, homologs and orthologs include, but are not limited to: nucleic acids that are complementary or at least partly complementary to any one of SEQ ID NOS:8-11 and 39; nucleic acids comprising nucleotide sequences varied to take account of codon sequence redundancy including codon-optimized variants of any one of SEQ ID NOS:8-11 and 39; and naturally occurring or allelic variants of any one of SEQ ID NOS: 8-11 and 39.

The invention also contemplates fragments of the isolated nucleic acids of the invention.

By “nucleic acid fragment” is meant a single- or double-stranded nucleic acid portion or sub-sequence of an isolated nucleic acid of the invention. The fragment may comprise a contiguous nucleotide sequence that constitutes at least 1%, 2%, 5%, 7%, 10%, 20%, 30%, 40%, 50%, 60%, 70%, 80%, 90%, 95% or 99% of the contiguous nucleotide sequence of an isolated nucleic acid of the invention. The fragment may comprise a contiguous nucleotide sequence of at least 6, 10, 15, 20, 30, 50, 80, 100, 200, 300, 400, 500, 600, 700, 800, 900, 1000, 1100, 1200, 1300, 1400 or 1500 contiguous nucleotides of an isolated nucleic acid of the invention.

Non-limiting examples of nucleic acid fragments are the open reading frames defined by residues 1-609 and residues 677-1606 of SEQ ID NO:11.

In certain aspects, the nucleic acid fragment may be a primer or a probe.

A “primer” is usually a single-stranded oligonucleotide, preferably having 9-60 contiguous nucleotides, which is capable of annealing to a complementary nucleic acid “target” and being extended in a template-dependent fashion by the action of a DNA polymerase such as Taq polymerase, RNA-dependent DNA polymerase or Sequenase™.

In particular embodiments, a primer may comprise at least 10, 12, 15, 18, 20, 25, 30, 35, 40, 45 or 50 contiguous nucleotides.

A “probe” may be a single or double-stranded oligonucleotide or polynucleotide that is capable of annealing to a nucleotide sequence of a target nucleic acid that is at least partly complementary to said probe.

Probes and primers may be labeled to facilitate detection. Labels include radioisotopes, fluorophores including fluorescence donor and acceptor molecules and enzymes for colorimetric or chemiluminescent detection, as are well understood in the art.

A “target” nucleic acid may be any nucleic acid that is detectable, bindable or otherwise capable of being annealed to by a primer or probe of the invention.

The invention also provides “genetic constructs” that comprise one or more isolated nucleic acids, fragments or variants of the invention.

As generally used herein, a “genetic construct” is an artificially created nucleic acid that incorporates, and/or facilitates use of, an isolated nucleic acid encoding an isolated M protein, fragment, variant or derivative of the invention.

In particular embodiments, such constructs may be useful for recombinant manipulation, propagation, amplification, homologous recombination and/or expression of said isolated nucleic acid.

As used herein, a genetic construct used for protein expression is referred to as an “expression construct”, wherein the isolated nucleic acid to be expressed is operably linked or operably connected to one or more regulatory sequences in an expression vector.

An “expression vector” may be either a self-replicating extra-chromosomal vector such as a plasmid, or a vector that integrates into a host genome.

In one aspect of the invention, the expression vector is a plasmid vector.

By “operably linked” or “operably connected” is meant that said regulatory nucleotide sequence(s) is/are positioned relative to the nucleic acid to be expressed to initiate, regulate or otherwise control expression of the nucleic acid.

Regulatory nucleotide sequences will generally be appropriate for the host cell used for expression. Numerous types of appropriate expression vectors and suitable regulatory sequences are known in the art for a variety of host cells.

One or more regulatory nucleotide sequences may include, but are not limited to, promoter sequences, leader or signal sequences, ribosomal binding sites, transcriptional start and termination sequences, translational start and termination sequences, splice donor/acceptor sequences and enhancer or activator sequences.

Constitutive or inducible promoters as known in the art are may be used and include, for example, tetracycline-repressible, IPTG-inducible, alcohol-inducible, acid-inducible and/or metal-inducible promoters.

In one aspect, the expression vector comprises a selectable marker gene. Selectable markers are useful whether for the purposes of selection of transformed bacteria (such as bla, kanR and tetR) or transformed mammalian cells (such as hygromycin, G418 and puromycin).

Suitable host cells for expression may be prokaryotic or eukaryotic, such as S. iniae, Escherichia coli (DH5α for example), yeast cells, SF9 cells utilized with a baculovirus expression system, or any of various mammalian or other animal host cells, without limitation thereto.

Introduction of expression constructs into suitable host cells may be by way of techniques including but not limited to electroporation, heat shock, calcium phosphate precipitation, DEAE dextran-mediated transfection, liposome-based transfection (e.g. lipofectin, lipofectamine), protoplast fusion, microinjection or microparticle bombardment, as are well known in the art.

In particular aspects, the invention provides a composition and/or method for treating an animal infected with S. iniae infection.

In one particular aspect, the invention provides a method of treating an animal infected with S. iniae, said method including the step of administering a composition of the invention to said animal to thereby prophylactically or therapeutically treating said S. iniae infection.

In another particular aspect, the invention provides a method of immunizing an animal against S. iniae, said method including the step of administering a composition of the invention to said animal to thereby immunize against S. iniae.

One particular feature of the S. iniae M proteins, or M-like proteins of the invention is that from 50 isolates tested, there was relatively limited sequence variation. Accordingly, the S. iniae M proteins, or M-like proteins, may be useful in eliciting cross-protective immune responses against a plurality of S. iniae strains.

Compositions and methods of the invention may suitably be practised in relation to any animal host susceptible to S. iniae infection, including dolphins and fish, although without limitation thereto.

Compositions may comprise isolated S. iniae M proteins or fragments thereof antibodies which bind S. iniae M proteins or fragments thereof isolated nucleic acids encoding S. iniae M proteins or fragments thereof and/or attenuated or inactivated S. iniae bacteria which have been induced to express S. iniae M proteins.

Compositions of the invention may be for veterinary or medical use and comprise a suitable carrier, diluent or excipient.

In one aspect of the invention, an “immunotherapeutic composition” is provided which, upon administration to an animal, provides, assists or elicits an immune response to S. iniae in an animal. Such a composition may be in the form of a vaccine.

Any suitable procedure is contemplated for producing immunotherapeutic composition and vaccines.

In one aspect, the invention provides passive or active immunization of animals against S. iniae infection.

Passive immunization is suitably provided through administration of an effective amount of an antibody or antibody fragment as hereinbefore described.

Active immunization may be achieved through administration of an effective amount of an M protein or immunogenic fragment thereof. Isolated M proteins and immunogenic fragments thereof may be in the form of recombinant proteins or chemically synthesized peptides.

Attenuated bacterial vaccines may be administered to animals, including fish and humans.

For example, attenuated bacteria may express one or more recombinant S. iniae M proteins or fragments thereof, or are attenuated S. iniae bacteria induced to express one or more endogenous S. iniae M proteins.

Attenuation or inactivation of bacteria, such as by heat, chemical treatment etc. is well known in the art.

The invention also provides nucleic acid vaccines that encode one or more isolated S. iniae M proteins or fragments thereof.

As used herein, the term “nucleic acid vaccine” includes within its scope “plasmid vaccines” and “viral vaccines”.

In one aspect, the nucleic acid vaccine is a plasmid DNA vaccine.

In general terms, by “carrier, diluent or excipient” is meant a solid or liquid filler, diluent or encapsulating substance that may be safely used in administration to an animal, such as a fish or a human. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, isotonic saline, pyrogen-free water, tricaine, wetting or emulsifying agents, bulking agents, coatings, binders, fillers, disintegrants, diluents, lubricants, pH buffering agents.

In the case of nucleic acid vaccines, the DNA expression vector may be delivered naked, or may be provided in the form of cationic lipid-DNA complexes, liposomes, calcium phosphate co-precipitates, adsorbed on microparticles, although without limitation thereto.

An immune response elicited by administration of a nucleic acid vaccine of the present invention may be enhanced using other nucleotide sequences.

Non-limiting examples of such other nucleotide sequences include immune-stimulating oligonucleotides having unmethylated CpG dinucleotides, or nucleotide sequences that code for other antigenic proteins or adjuvanting cytokines. The DNA can be present in naked form or it may be administered together with an agent facilitating cellular uptake (e.g. liposomes or cationic lipids).

For immunotherapeutic compositions and vaccines, an adjuvant may be included.

Suitable adjuvants include, but are not limited to, surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminum phosphate, aluminum hydroxide or alum; lymphokines, QuilA and immune stimulating complexes (ISCOMS).

The effective dosage of a composition or vaccine may vary depending on the size and species of the subject, and according to the mode of administration. The optimal dosage can be determined through trial and error by a doctor, veterinarian or aquaculture specialist.

For fish, vaccines may comprise between 0.01 and 0.5 mg, preferably between 0.025 and 0.25 mg or more preferably between about 0.05 and 0.2 mg of protein in a single dosage.

A suitable dosage range for nucleic acid vaccines may be as low as picogram, or as high as mg quantities, but is normally from about 0.01 to 100 μg, preferably 0.1 μg to 50 μg per unit dose, more preferably about 1 μg to 25 μg, and most preferably about 5 μg to 10 μg per unit dose.

Due to possible stress suffered by fish in response to vaccination, it is preferred that the vaccine is provided as a single shot vaccine, in single dosage form.

For injectable vaccines, a single dosage unit is suitably 0.025 to 0.5 ml, preferably 0.04 to 0.2 ml, about 0.05 to 0.1 ml, in volume.

In aspects relating to fish, compositions and vaccines may prepared as liquid solutions, attenuated bacteria, emulsions or suspensions for injection or by immersion delivery in water. Solid (e.g. powder) forms suitable for dissolution in, or suspension in, liquid vehicles, or for mixing with solid food, prior to administration may also be prepared. The composition or vaccine may be lyophilized, optionally freeze-dried, in a ready to use form for reconstitution with a sterile diluent. For instance, lyophilized vaccine may be reconstituted in 0.9% saline (optionally provided as part of the packaged vaccine product).

Nucleic acids are particularly suited to lyophilisation due to the stability and long shelf-life of the molecules. Alternatively, the composition or vaccine may be provided in a saline solution. Liquid or reconstituted forms of the vaccine may be diluted further in a small volume of water (e.g. 1 to 10 volumes) before addition to a pen, tank or bath for administration to fish by immersion. The vaccine compositions of the invention may be administered in a form for immediate release or extended release.

For nucleic acid-based therapy or vaccination in fish, one may select transcriptional regulatory sequences endogenous to the fish to be vaccinated. For instance, endogenous cytokine or actin gene promoters may be considered, or other regulatory sequences may be derived from fish DNA viruses. Non-limiting examples of DNA vaccination as applied to fish are provided in U.S. Pat. No. 5,780,448, U.S. Patent Publ. No. 20050163795, U.S. Patent Publ. No. 20060073167 and U.S. Patent Publ. No. 20050261227.

In embodiments relating to treatment and/or immunization of fish, compositions may also be administered orally or by immersion of the fish in a dilute composition comprising the immunogenic M protein or fragment thereof.

Oral fish compositions include within their scope fish feeds and/or supplements that may be in pellet, granule, liquid, flake or powder form comprising an isolated S. iniae M protein or immunogenic fragment thereof.

Compositions, vaccines, treatment and/or immunization methods relevant to fish may be particularly useful in the context offish farming, fish hatcheries and other commercial fisheries applications. However, it will also be appreciated that the invention may have applications in conservation of wild fish populations where S. iniae infections have adverse affects upon native or other wild fish populations.

In certain aspects, the invention provides compositions and/or methods for treating S. iniae infections of animals other than fish.

In one particular aspect, the invention provides compositions and/or methods for treating S. iniae infections of humans.

For humans, dosages may be calculated empirically according to the age, weight sex and general health of the human.

By way of example only, protein dosages in the range 0.1 to 1 mg per 70 kg body weight, or preferably about 0.3 to 0.5 mg per 70 kg body weight may be effective.

Any suitable route of administration may be employed for a human patient according to the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms may include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion.

Compositions and vaccines suitable for administration to humans may be produced by procedures such as described in New Generation Vaccines (1997, Levine et al., Marcel Dekker, Inc. New York, Basel, Hong Kong), by way of example only.

Compositions and vaccines of the invention may be administered to humans in the form of attenuated bacterial vaccines where the bacteria express one or more recombinant S. iniae M proteins or fragments thereof. Non-limiting examples of attenuated bacteria include Salmonella species, for example Salmonella enterica var. Typhimurium or Salmonella typhi. Alternatively, other enteric pathogens such as Shigella species or E. coli may be used in attenuated form. Attenuated Salmonella strains have been constructed by inactivating genes in the aromatic amino acid biosynthetic pathway (Alderton et al., Avian Diseases 35 435), by introducing mutations into two genes in the aromatic amino acid biosynthetic pathway (such as described in U.S. Pat. No. 5,770,214) or in other genes such as htrA (such as described in U.S. Pat. No. 5,980,907) or in genes encoding outer membrane proteins, such as ompR (such as described in U.S. Pat. No. 5,851,519).

Alternatively, attenuated bacterial vaccines may include S. iniae bacteria induced to express one or more endogenous S. iniae M proteins or fragments thereof.

Human compositions and vaccines may further comprise a carrier.

Non-limiting examples of carriers include thyroglobulin; albumins such as human serum albumin, toxins, toxoids or any mutant cross reactive material (CRM) of the toxin from tetanus, diptheria, pertussis, Pseudomonas, E. coli, Staphylococcus, and Streptococcus, polyamino acids such as poly(lysine:glutamic acid), influenza; Rotavirus VP6, Parvovirus VP1 and VP2, hepatitis B virus core protein, hepatitis B virus recombinant vaccine and the like. Alternatively, a fragment or epitope of a carrier protein or other immunogenic protein may be used. For example, a T cell epitope of a bacterial toxin, toxoid or CRM may be used.

In humans, compositions and/or vaccines of the invention may be administered in multivalent form in combination with antigens of organisms inclusive of the pathogenic bacteria H. influenzae and other Haemophilus species, M. catarrhalis, N gonorrhoeae, E. coli, S. pneumoniae, etc.

In other particular aspects, the invention provides diagnostic methods to detect S. iniae infections in animals.

In one aspect, the invention provides a method of determining whether an animal is, or has been, exposed to S. iniae bacteria or components thereof, said method including the step of determining whether a biological sample obtained from said animal comprises an isolated S. iniae M protein or fragment, variant or derivative thereof, wherein the presence of said isolated S. iniae M protein or fragment, variant or derivative thereof indicates that said animal is, or has been, exposed to S. iniae bacteria or molecular components thereof.

In another aspect, the invention provides a method of determining whether an animal is, or has been, exposed to S. iniae bacteria or components thereof, said method including the step of determining whether a biological sample obtained from said animal comprises an isolated nucleic acid of the invention, wherein the presence of said isolated nucleic acid indicates that said animal is, or has been, exposed to S. iniae bacteria or molecular components thereof.

In yet another aspect, the invention provides a method of determining whether an animal is, or has been, exposed to S. iniae bacteria or components thereof, said method including the step of determining whether a biological sample obtained from said animal comprises an antibody or antibody fragment that binds an isolated S. iniae M protein or fragment thereof, wherein the presence of said antibody or antibody fragment indicates that said animal is, or has been, exposed to S. iniae bacteria or molecular components thereof.

The invention also provides a diagnostic kit and/or diagnostic composition for detecting S. iniae bacteria, molecular components thereof or an antibody which binds the M protein of the invention or a fragment thereof.

Diagnostic kits and/or compositions may be suitable for nucleic acid-based or protein-based detection and comprise one or more diagnostic agents, such as one or more antibodies, M proteins or fragments thereof, nucleic acid probes and/or primers to facilitate detection of antibodies to S. iniae M proteins, S. iniae M proteins, encoding nucleic acids and/or fragments thereof in a biological sample.

Detection of nucleic acids may employ teachniques well known in the art, including but not limited to, Northern hybridization, Southern hybridization, nucleotide sequence amplification, nucleic acid array-hybridization, mass spectrometry of primer extension products, DNA sequencing and the like.

Detection methods may further include a step of nucleotide sequence amplification of an S. iniae M protein-encoding nucleic acid or fragment thereof.

As used herein, a “nucleic acid sequence amplification technique” includes but is not limited to polymerase chain reaction (PCR) strand displacement amplification (SDA); rolling circle replication (RCR); nucleic acid sequence-based amplification (NASBA) as for example described by; ligase chain reaction (LCR); Q-βreplicase amplification; and helicase-dependent amplification.

In one embodiment, the nucleotide sequence amplification technique is PCR.

Accordingly, in one particular embodiment, the invention provides a method of nucleic acid sequence amplification including the step of using a thermostable DNA polymerase and one or more primers to amplify an S. iniae M protein-encoding nucleic acid or fragment thereof present in a nucleic acid sample obtained from an animal.

Non-limiting examples of PCR primers that may be useful in diagnostics are provided in Table 1. However, it will be readily appreciated that a skilled person may design and produce other PCR primers based on one or more nucleotide sequences set forth in any one of SEQ ID NOS: 8-11.

PCR amplification products may conveniently be detected using a labeled DNA probe complementary to any one of SEQ ID NOS: 8-11.

Accordingly, the invention contemplates a diagnostic kit comprising:
(a) one or more single stranded DNA primers capable of annealing to an S. iniae M protein-encoding nucleic acid or fragment thereof; and
(b) a DNA probe capable of hybridizing to one or more PCR amplification products PCR amplified from an S. iniae M protein-encoding nucleic acid or fragment.

The kit may further comprise a thermostable DNA polymerase.

The invention also contemplates protein-based detection of isolated S. iniae M proteins.

Protein-based detection may be performed using an antibody or antibody fragment as hereinbefore described.

Suitable methods may employ immunoblotting, immunoprecipitation, ELISA, protein arrays, protein profling, 2D electrophoresis, mass spectroscopy, protein sequencing, radioimmunoassays and radioligand binding, although without limitation thereto.

Accordingly, the invention contemplates a diagnostic kit comprising:
(a) an antibody or antibody fragment capable of binding an isolated S. iniae M protein or fragment thereof;
(b) one or more detection reagents to facilitate detection of said antibody or antibody fragment bound to said isolated S. iniae M protein or fragment thereof.

Detection reagents may include secondary antibodies labelled for colorimetric, fluorimetric or luminescent detection and an appropriate substrate.

Alternatively, the antibody or antibody fragment capable of binding an isolated S. iniae M protein or fragment thereof may be directly labelled.

Non-limiting examples of antibody labels are as hereinbefore described.

It is also contemplated that antibodies produced by an animal in response to S. iniae infection may also be detected as an indicator as to whether an animal is, or has been, exposed to S. iniae bacteria or molecular components thereof.

The presence of such antibodies may be detected using any of the aforementioned protein detection methods, for example by ELISA and immunoblotting methods.

For the purposes of diagnosis, biological samples from fish or other animals may suitably be in the form of blood or biopsy samples such as from kidney, spleen, heart, brain, muscle, or other tissues.

So that the present invention may be more readily understood and put into practical effect, the skilled person is referred to the following non-limiting examples.

### EXAMPLES

### Isolation and Characterization of S. iniae M Protein

### Experimental Procedures

S. iniae Strains and Culture Conditions

Veterinary laboratory isolates of S. iniae taken from infected fish (Lates calcarifer) were used in this study. Strains in 20% glycerol that were stored at −80° C. were grown overnight at 37° C. on Columbia agar base containing 5% defibrinated sheep blood.

### Recombinant DNA Techniques

S. iniae genomic DNA was extracted from freshly-grown cells using an enzymatic lysis method (Pruksakorn et al., 2000, J. Clin. Microbiol. 38:125).

PCR Amplification of S. iniae emm-Like (sim) Genes and Cloning

Each 50 μL PCR tube contained 5 μL of 10×Tth plus buffer, 1 μL dNTP's (4×2.5 mM of each of dATP, dCTP, dGTP and dTTP; Biotech International Ltd., Australia), 200 ng of ALL MF and ALL MR primers (Table 1), 0.5 U of Tth plus DNA polymerase (Biotech International Ltd., Australia), 3 μL of 25 mM magnesium chloride, and the balance made up with sterile Milli-Q water. Thermal cycling parameters in an Eppendorf Mastercycler Gradient EPS (Eppendorf, Hamburg, Germany) were denaturation for 2 mins at 94° C. followed by 35 cycles of 50° C. for 1 min, 72° C. for 2 min, and 94° C. for 15 s with a final extension cycle of 50° C. for 1 min and 72° C. for 10 min. The resultant PCR products were visualised after electrophoresis in a 1% w/v agarose gel containing 0.5 μL of 10 mg/mL ethidium bromide solution in 1×TAE as the electrophoresis buffer.

Desired bands were excised from the gel and extracted from the agarose with the MegaSpin Agarose Gel Extraction Kit (Intron biotechnology, Korea). Purified PCR products were ligated into PCR4-TOPO by TA cloning using a commercial kit (Invitrogen, Melbourne, Australia). Competent cells (TOP 10; Invitrogen, Melbourne, Australia) used in cloning experiments were cultured on Luria-Bertani Agar (LB Agar; Sigma, Castle Hill, Australia) supplemented with 100 μg/mL ampicillin. X-gal (40 μA of 20 mg/ml) was spread onto LB agar plates for blue-white discrimination. Clones were grown at 37° C. overnight. Clones were picked using a sterile disposable loop and plated onto LB agar. These confirmed white clones were also used for screening by direct lysis PCR and for storage.

### Direct Lysis PCR of Clones and Sequencing.

The reaction volume was scaled down to 25 μL, with the same reaction conditions as above. Use of plasmid-specific primers SP6 and T7 (Table 1) enabled the sim gene inserts to be amplified. Agarose gel electrophoresis allowed determination of clones with inserts, which were then subjected to plasmid extraction using a commercial kit in accordance with the manufacturers instructions (Invitrogen, Melbourne, Australia) for sequencing with plasmid primers SP6 and T7 (Table 1). Sequencing of the full length of the sim genes was initially facilitated with the primer SIM F (Table 1).

### Genome Walking and Sim Gene Diversity.

Sequences upstream and downstream of the sim gene were obtained by genome walking according to the manufacturer's instructions (Genome Walker Kit, Clontech, Mountain View, Calif.). Gene specific primers used for genome walking upstream of the sim gene were SIM WR and SIM W2R (Table 1) and SIM WF and SIM W3F (Table 1). Resultant PCR products were gel-purified and ligated into TOPO vector PCRII (Invitrogen, Melbourne, Australia). Sequencing was carried out with SP6 and T7 primers on recombinant clones. This allowed primers to be designed (PRE SIM and POST SIM; Table 1) for the amplification of the sim genes and the surrounding spacers (using the same reaction component concentration as above, but with a 65° C. annealing temperature and use of proofreading Prime STAR DNA polymerase (Takara, Shiga, Japan) to reduce the likelihood of incorporation errors) from additional strains to examine sim gene diversity. The PRE SIM primer is located in the multigene regulator gene (mgrX) and includes the first 36 nucleotides of the mgrX gene in the amplicons and the POST SIM primer will amplify the last 75 nucleotides of the putative tellurite/toxic anion resistance gene (telX). These PCR products were directly sequenced with the following primers: PRE SIM, SIM R, SIM 2R, SIM F, SIM 2F, SIM 3F, SIM W2R, and POST SIM (Table 1). Additionally, primers M141 F and SIM3 F RC were used to generate sequence data from isolate QMA0141.

### Expression of M-Like Proteins

M-like proteins were expressed using the Champion pET system (pET101/D-TOPO; Invitrogen, Melbourne, Australia) and expressing in E. coli BL21 Star (DE3) One Shot chemically competent cells (Invitrogen, Melbourne, Australia) according to the manufacturer's instructions. Representatives of the different sim sequevars recovered were expressed according to the manufacturer's instructions. Representative isolates of the different sim genes QMA0072, QMA0076, and QMA0141 were expressed as proteins.

### Fibrinogen Binding Assay

Human fibrinogen (Sigma, Castle Hill, Australia) was labelled using the EZ-Link Sulfo-NHS Biotinylation Kit (Pierce, Rockford, Il.). Unincorporated biotin was removed using Micro-Spin G-25 columns (GE Healthcare Biosciences, North Ryde, Australia). SDS-PAGE separation of IPTG-induced cellular lysates were blotted onto PVDF membranes and detected using streptavidin-alkaline phosphatase (Pierce, Rockford, Il.) and colour developed using 1-Step NBT-BCIP (Pierce, Rockford Il.).

### Isolation of Head Kidney and Peritoneal Cells.

Macrophages from casein stimulated peritoneal cavity were harvested, purified and maintained as described previously (Do Vale et al., 2002). Briefly, for stimulation of the peritoneum, barramundi (Lates calcarifer) (300 g) were anaesthetisized with Aqui-S (Aquatic Diagnostic Services, Wilston, Australia) in accordance with the manufacturers instructions, and then injected with 1 ml of 12% casein (sterile, in phosphate buffered saline, PBS) into their peritoneal cavity 24 hours before collection of macrophages. Prior to the isolation of peritoneal macrophages, fish were euthanised with overdose Aqui-S and then exsanguinated by cutting the ventral aorta. An aliquot (5 ml) L-15 medium containing 2% Foetal Bovine Serum (FBS, Invitrogen, Melbourne, Australia), 1% penicillin/streptomycin (P/S) (Invitrogen, Melbourne, Australia), and 10 μg·ml⁻¹ heparin (Sigma, Castle Hill, Australia) was injected asceptically into the peritoneal cavity using a syringe fitted with a 25 G needle. The body cavity was then massaged for 30 sec to disperse the medium and the lavage containing leucocytes was withdrawn using 19 G syringe very carefully to prevent bleeding. The suspensions of peritoneal cells were then layered onto a discontinuous (34%/51%) Percoll density gradient and centrifuged at 450 g for 25 min at 4° C. The band lying at the interface was collected and washed twice with L-15 medium containing 1% FBS and 1% penicillin/streptomycin (P/S). Concentration of viable cells was determined by Trypan blue exclusion. The cells were seeded in microtiter plates at the concentration of 10⁷ cells ml⁻¹ in L-15 medium with 1% FBS and 1% P/S. Cell populations were allowed to adhere for 2 hours at 28° C. and then washed twice with L-15 medium to remove the unattached cells. The adhered cells were maintained in L-15 with 1% FBS and 1% P/S at 28° C. For the present assay, 24 hour cultures were used.

### Induction of Luminol-Amplified Chemiluminescence

Streptococcus iniae strains QMA0072, QMA0076, and QMA0141 were used for this assay. An OD₆₀₀ 1.5 of each strain were pre-incubated with either PBS, 5 μg ml⁻¹ BSA in PBS, or 5 μg ml⁻¹ fibrinogen in PBS for 30 minutes at 37° C. (Welch, 1980). The cells were then washed twice with HBSS and resuspended in HBSS.

The respiratory burst activity of the head kidney cells following phagocytosis was determined using protocols described previously (Nikoskelainen et al., 2005) with some modifications. Briefly, one-day old head kidney cells were stimulated with either un-treated or treated S. iniae at a multiplicity of infection (MOI) of 100 (bacteria/macrophage ratio 1:100). The respiratory burst activity was initiated by the addition of 30 μl of the required bacterial suspension to macrophages in microtitre plates (1×10⁵ cells per well). To each well, 10 μl of 10 mM luminol in 0.2 M borate buffer pH 9.0 and 280 μl HBSS pH 7.4 were also added to get a final volume of 300 μl. The chemiluminescence (CL) emissions of the phagocytes were measured in a luminometer/fluorometer (BMG Fluostar Optima, BMG Labtech, Offenberg, Germany) every 3 minutes for 3 hours at 27° C.

### Results

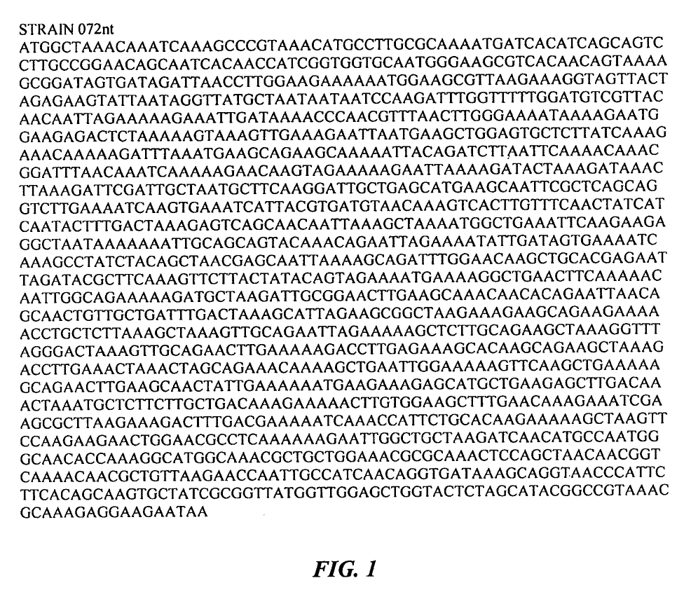

FIG. 1 shows S. iniae strain QMA72 M protein-encoding nucleotide sequence (SEQ ID NO:8).

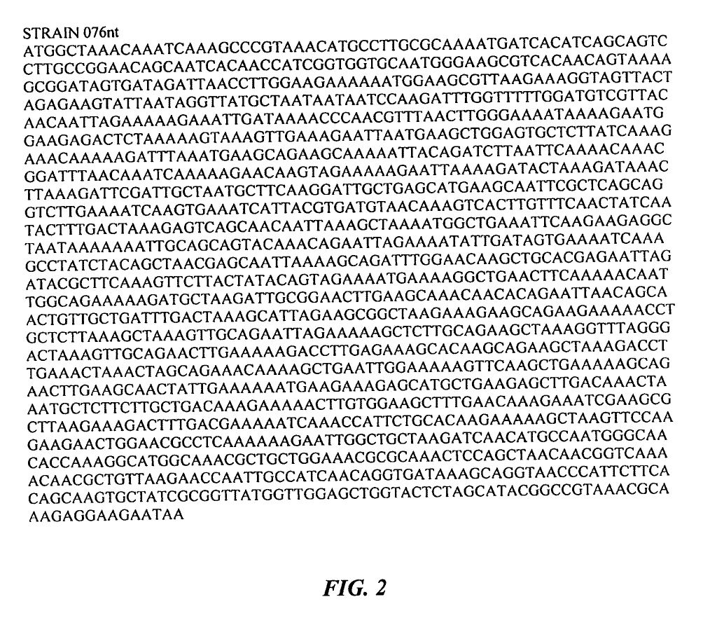

FIG. 2 shows S. iniae strain QMA76 M protein-encoding nucleotide sequence (SEQ ID NO:9).

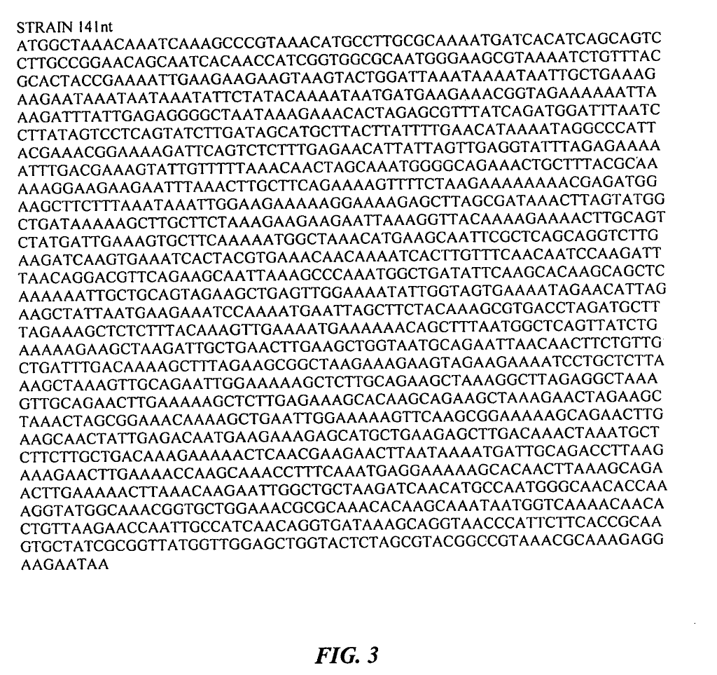

FIG. 3 shows S. iniae strain QMA141 M protein-encoding nucleotide sequence (SEQ ID NO:10).

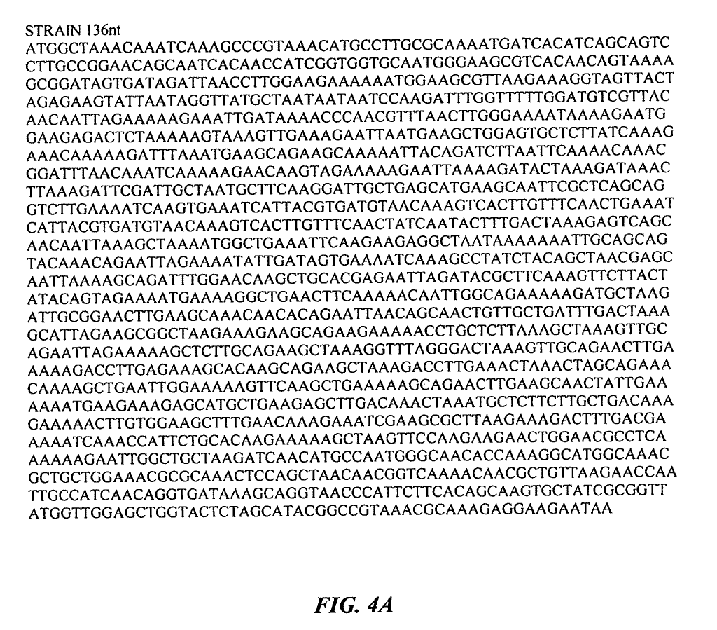

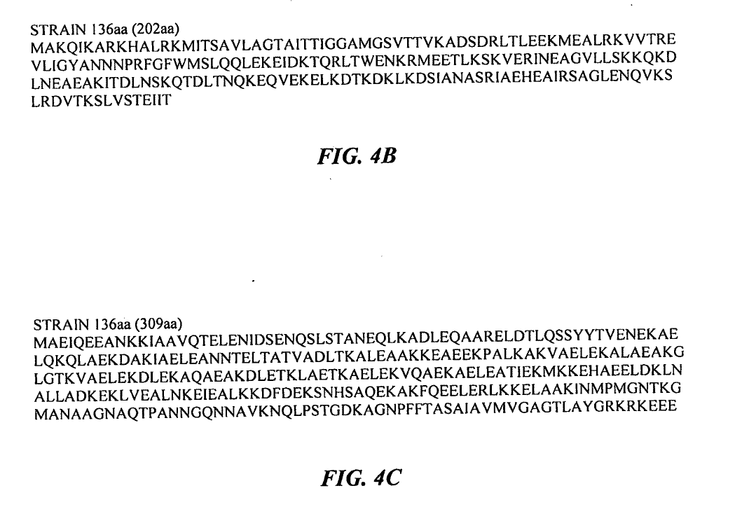

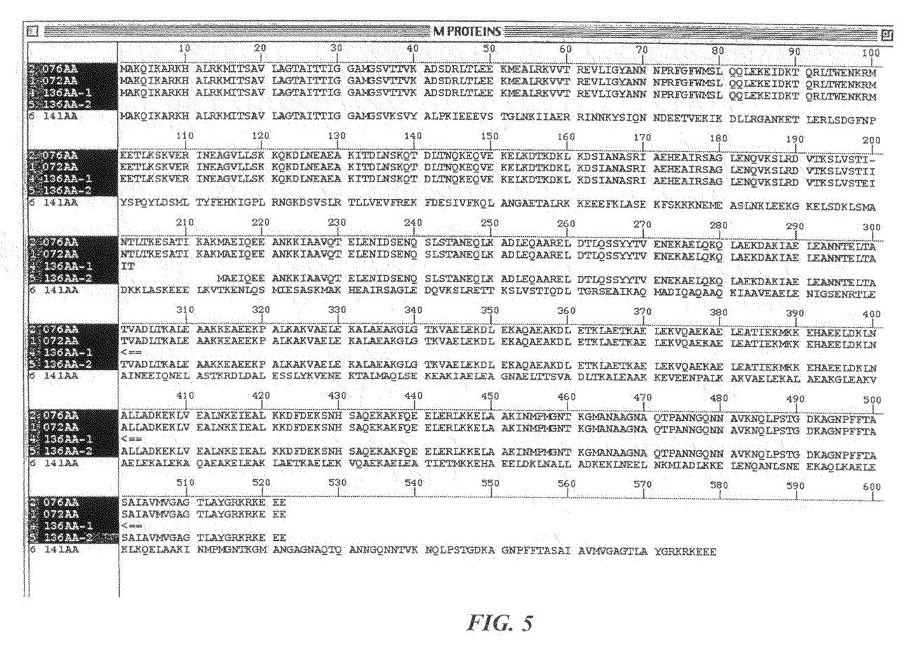

FIG. 4 shows S. iniae strain QMA136 M protein-encoding nucleotide sequence (SEQ ID NO:11), N-terminal fragment amino acid sequence (SEQ ID NO:6) and C-terminal fragment amino acid sequence (SEQ ID NO:7).

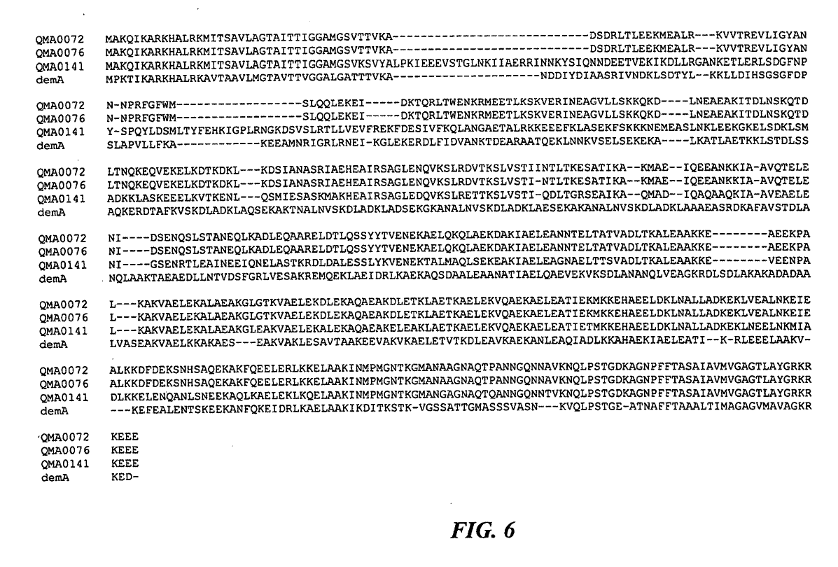

Alignments of QMA72 M protein amino acid sequence (SEQ ID NO:3), QMA76 M protein amino acid sequence (SEQ ID NO:4) and QMA141 M protein amino acid sequence (SEQ ID NO:5) is shown in FIG. 5.

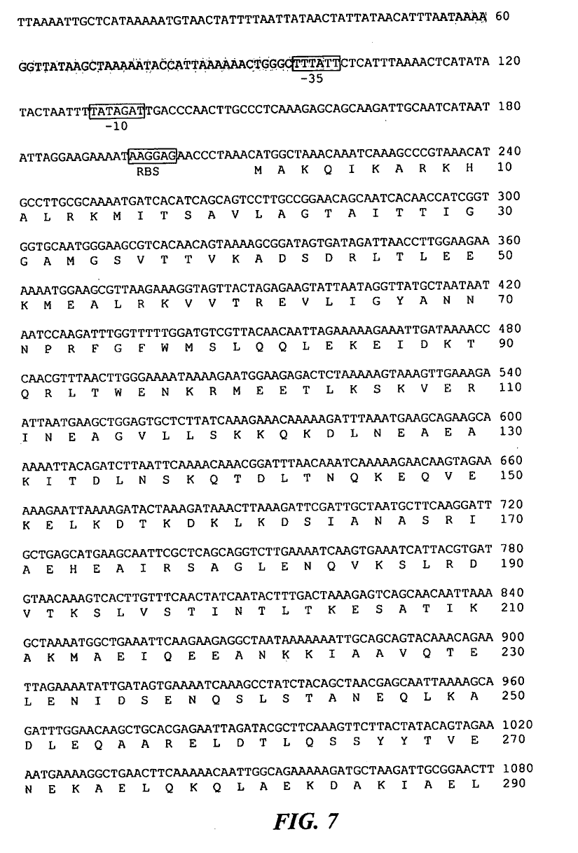

An alignment with the demA gene product is shown in FIG. 6.

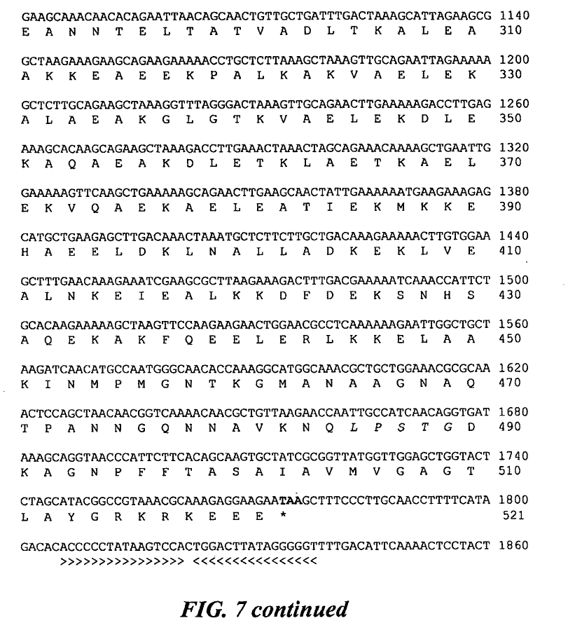

FIG. 7 shows an S. iniae SimA gene sequence (SEQ ID NO:39) with 5′ and 3′ untranslated nucleotide sequence and encoded M protein.

Using the ALL M primer pair (Table 1) allowed sequencing of the full length of the simA (Streptococcus iniae emm-like) gene. Primers for genome walking were designed to determine the true sequence of the gene for the extreme 5′ and 3′ end nucleotides since the ALL M primer pair introduced artefactual nucleotides (spacer region and encoding amino acids MA at the 5′ end and KRKEEN (SEQ ID NO:13) at the 3′ end) to the initially deduced sequence. The full length of the simA gene is 1566 bp encoding a 521 amino acid protein in most strains (FIG. 4), however, the protein for isolate QMA0072 had one amino acid insertion, and the sim gene for isolate QMA0141 (encoded by simB) was 579 amino acids (Table 2). The respective molecular masses of the proteins (with signal peptides) for isolates QMA0072, QMA0076, and QMA0141 were 57 589, 57 467, and 63 667 Da respectively. The mature proteins have molecular masses of 53 416, 53 303, and 59 446 Da respectively. There were changes to the amino acid sequence at the 3′ end of the nucleotide sequence encoding for KRKEEE (SEQ ID NO:14).

A probable ribosomal binding site with sequence AAGGAG (SEQ ID NO:15) was found 11 bases upstream of the start of transcription of the simA gene (FIG. 7). A putative Mgx binding site 45 nucleotides in length was found with a recognition site very similar to that found in the promoter regions of the emm and scpA genes in GAS; FIG. 7).

Diversity of Sim Genes in S. iniae.

Sequencing of the sim gene from strain QMA0076 revealed that it was distinct from the other known M and M-like protein types from S. pyogenes, S. suis, S. equi subsp. equi, S. dysgalactiae, and S. uberis. Sequence alignment of the different types of sim gene products from S. iniae with their closest matching counterpart, the demA gene product from S. dysgalactiae, are presented in FIG. 6.

By using the PRE SIM and POST SIM primer pair, an expected PCR product of 2048 by was produced. This was the most common PCR product from the majority of 50 isolates tested. For isolate QMA0072, which had an insertion in the simA gene, a PCR product of 2051 bp was produced. QMA0141 resulted in a larger product of 2180 bp. The nucleotide and amino acid sequences for isolates QMA0076 and QMA0072 were identical except for the insertion of three nucleotides (1 aa residue). The length of the intergenic spacer regions either side of the simA gene in all isolates is the same size. The gene sequence for isolate QMA0141 was most divergent having 100% amino acid residue identity for the first 35 residues and 100% similarity for the first 41 residues. This also confirms the theoretical position of where the signal sequence is cleaved to produce the mature M protein (FIG. 7). This gene was approximately 10% larger than the simA gene and so was designated simB. Thus, the same genetic organization is present in all of the isolates tested.

### M Protein Sequence Analysis

The N-terminal of the major M protein type from isolate QMA0076 has a probable signal sequence cleavage site between amino acids 41 and 42 resulting in a mature protein of 480 amino acids with a molecular mass of ˜53 kDa (FIG. 6). For isolate QMA0141, the signal peptide cleavage point is also between residues 41 and 42 resulting in a mature protein of 538 residues with a molecular mass of ˜59 kDa (FIG. 6). In the C terminal end of all of the M proteins is the conserved Gram positive cell wall anchor motif LPSTG (SEQ ID NO:16; Vasi et al., 2000, Infect. Immun. 68 294). The N-terminal region of the M protein types had very similar signal peptides, but the first residues of the mature protein differed significantly while there was a high level of conservation at the C terminal ends of the proteins. This indicates that selective pressures are being exerted upon the exposed N terminal parts of the protein.

Analysis by the Garnier algorithm (Garnier et al., 1978, J Mol Biol 120 97) for isolate QMA0076 predicted that 77.2% of the protein was alpha-helical. For isolates QMA0072 and QMA0141, the predicted values were 77.1% and 77.4% respectively for their M proteins.

Analysis by the COIL program (Lupas et al., 1991, Science 252 1162; Lupas, 1996, Methods Enzymol 266 513) with a window length of 28 predicted that the protein from isolate QMA0076 (with signal sequence) has two coiled-coil segments from residues 79-174 and 181-454 with a probability of 1.00 for each segment. This is a similar result to that of Vasi et al., supra who identified M-like proteins from S. dysgalactiae. By weighting the first and fourth residues in the heptad repeats a probability of 1.00 was gained for residues 91 to 449. Similarly, for isolate QMA0072 two coiled-coil segments from residues 79-174 and 196-455 with a probability of 1.00 for each segment were predicted.

Weighting the first and fourth residues in the heptad repeats gave a prediction with a probability of 1.00 for two segments from residues 91-186 and 213-450.

The M protein from isolate QMA0141 showed a probability of 1.00 that there was a coiled-coil structure from residues 154-512 with a weighted heptad, and coiled-coils at residues 151-231 and 238-512 when an unweighted heptad was used.

PAGE Analysis of Expressed M proteins and M Protein Fragments.

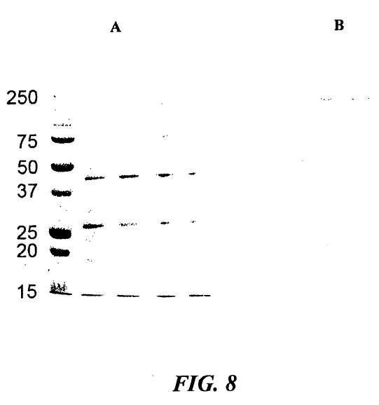

Analysis by SDS-PAGE of non-reduced expressed proteins (FIG. 8) showed that there were bands present which were different to that contained in the control E. coli lysate. Prominent protein bands were present at ˜230 kDa from isolates QMA072 and QMA076; but not for isolate QMA0141. Western blotting was used to detect the fibrinogen binding proteins from these lysates.

Fibrinogen Binding by Expressed M proteins

Binding of biotinylated fibrinogen followed by streptavidin detection in Western blots of expressed proteins from E. coli lysates showed that the M proteins from isolates QMA0072, and QMA0076 appeared as monomeric and tetrameric units (QMA0072 and QMA0076 only) at ˜230 kDa and ˜57 kDa as expected. The formation of tetrameric forms has been observed in M proteins from other species (Meehan et al., 1998, Microbiology 144 993). The M protein from isolate QMA0141 at ˜64 kDa does not appear to bind fibrinogen as strongly as that from isolates QMA0072 and QMA0076 (FIG. 8).

The two peptides expressed from isolate QMA0136 (202 amino acid SEQ ID NO:6 and 309 amino acid SEQ ID NO:7) did not bind fibrinogen (data not shown). The region where fibrinogen binding might occur is in the region where both the gene types (72 and 136) have their respective insertion sequences compared to isolate QMA76 (see FIG. 5). These insertions occur in the protein between amino acid residues 190-220.

### Effect of Fibrinogen Binding on Activation of Barramundi Peritoneal Leucocytes

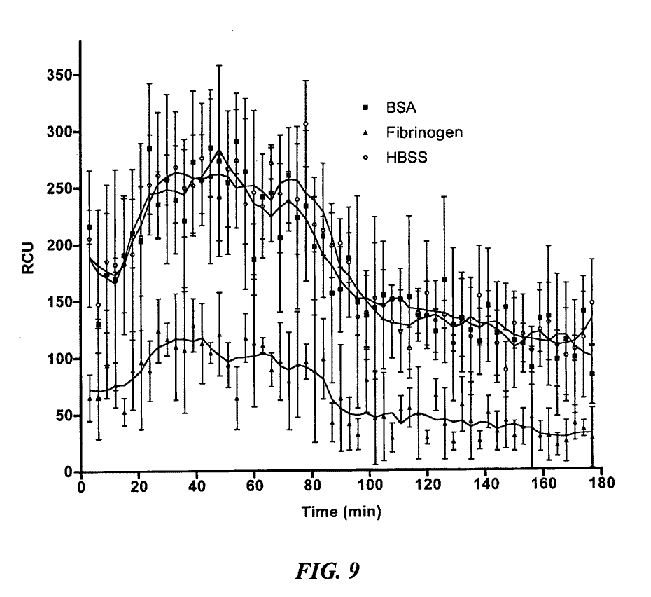

To investigate the role of fibrinogen binding by the M proteins in S. iniae, the effect of pre-incubating S. iniae QMA0072 in fibrinogen on phagocytosis and respiratory burst in peritoneal leucocytes was investigated. Fibrinogen binding by QMA0072 significantly decreased subsequent phagocytosis-induced respiratory burst in barramundi peritoneal leucocytes when compared to controls pre-incubated with the same concentration of BSA or in PBS alone (FIG. 9).

### Discussion

The sim genes and neighbouring genes from S. iniae have been described. As evidenced by Western blotting of recombinant M proteins from S. iniae expressed in E. coli, M proteins from isolate QMA0076 appear to bind the most fibrinogen, with that of isolate QMA0072 (which has one amino acid insertion) binding marginally less fibrinogen. The M protein from isolate QMA0141 bound very little fibrinogen following transfer to PVDF membrane. The observance of a slight decrease in the ability of isolate QMA0072's expressed protein to bind fibrinogen relative to that of isolate QMA0076 may be due to the one residue insertion after the intercoil region (in the second coiled region) disrupting the optimum binding ability of the coils. It is interesting to note that all S. iniae M protein sequences (except the QMA136 C-terminal fragment), regardless of size or divergence, contain the peptide sequence HEAIRSAGLE (SEQ ID NO:17) that connects the two coils. Differences in fibrinogen binding abilities may therefore be attributed to sequence variations in the coiled regions either side of this peptide sequence. Conservation of this peptide indicates that it is likely to have a critical role in binding of blood components such as immunoglobulins (Geyer et al., 1999, FEMS Immunol Med Microbiol 26 11). The role of this peptide sequence in the binding of fibrinogen and other blood components needs to be investigated further.

The ability to form tetrameric conglomerations may also be due to the coiled regions interacting with one another since the apparent molecular mass of ˜230 kDa has also been observed with a fibrinogen binding protein from S. equi subsp. equi (Meehan et al., 1998, supra). It is interesting to note that the ability of the M protein from isolate QMA0141, which had a much larger molecular weight, did not bind fibrinogen as efficiently as the major type or form a polymeric conglomeration. Given that other M and M-like proteins are able to bind other blood proteins, the ability of these proteins to bind them also cannot be discounted.

It has been shown that members of the M protein family contribute to virulence of Streptococcus sp., have the ability to bind blood components other than fibrinogen, and have a role in resistance to phagocytosis (Podbielski et al., 1996, Mol Microbiol 19 429; Meehan et al., 1998, supra; Thern et al., 1998, J Immunol 160 860; Meehan et al., 2000a, FEMS Microbiol Lett 190 317; Meehan et al., 2000b, Microbiology 146 1187; Meehan et al., 2001, Microbiology 147: 3311; Courtney et al., 2006, Mol Microbiol 59: 936). The present results support this observation, indicating that binding of fibrinogen by S. iniae reduced phagocytosis and subsequent respiratory burst activity in fish macrophages. Moreover, an earlier study indicated proteins with a similar apparent molecular weight to those reported in this study are capable of binding trout immunoglobulins in reverse orientation (i.e. by the Fc region) (Barnes et al., 2003, Fish Shellfish Immunol. 15 425). The M protein is a major virulence factor and vaccine candidate in GAS (Hu et al., 2002, Infect Immun 70 2171; Batzloff et al., 2006, Immunol Res 35 233; Olive, 2007, Curr Opin Mol Ther 9 25; Shaila et al., 2007, Vaccine 25 3567) and future work is directed towards investigating the potential of a vaccine against S. iniae infection in farmed fish based on these intriguing proteins.

### Vaccination with Recombinant S. iniae M Protein and DNA Vaccine Construct

### Experimental Procedures

### Experimental Animals and Husbandry

Juvenile barramundi, mean weight 12 g, were obtained from Ecofish Pty Ltd, Caloundra, Queensland. Fish were stocked at 80 fish per tank in five 300 litre food-grade circular plastic tanks containing 250 L brackish water (5 ppt) prepared from marine salts (Ocean Nature, Aquasonic, NSW) and RO filtered water. Eight tanks were connected to a large recirculating system with two 500 L sumps, with biofiltration and a separate protein skimmer. Air was supplied to each tank by compressors on a power supply independent to the supply driving the recirculation pumps (FIG. 1). Water was recirculated at 1800 L per hour and biofilters were established for 4 weeks prior to stocking with fish. Fish were acclimated for 14 days prior to vaccination and fed twice daily at 6 am and 6 pm with a commercial pelleted diet (Barramundi 4 mm floating, Ridley Aqua Feeds, Narangba, Qld). Water changes (200 L) were made as required, approximately twice per week. A 12 hour daylight cycle was maintained for the duration of the trial. Water temperature was recorded daily and maintained at 29° C.±1° C. via the aquarium room air conditioner. Ammonia, nitrite and nitrate were assayed daily using commercial kits (Aquasonic, NSW) and pH was recorded by taking a 5 ml water sample and recording on a laboratory pH metre.

### Preparation of Vaccines

Two vaccines were trialled: one a recombinant vaccine and the other a DNA vaccine. The gene used in each vaccine was simA derived from Streptococcus iniae which encodes the SiM protein, a virulence factor, as described by Baiano et al., 2008. The simA gene was PCR amplified from S. iniae strain QMA0076 which houses the dominant sim gene type from a global survey of S. iniae isolates and used in the production of the two vaccines.

### Recombinant Protein Vaccine

Recombinant SiM protein was produced for vaccination of barramundi by cloning the simA gene generated with primers ESIM 20F and ESIM 1542R (Table 3) and expressing it using the Champion pET system (pET101/D-TOPO; Invitrogen, Melbourne, Australia) in E. coli BL21 Star (DE3) One Shot chemically competent cells (Invitrogen, Melbourne, Australia) according to the manufacturer's instructions. Briefly, clones containing the insert in the correct orientation were grown overnight at 37° C. in 10 mL LB broth+100 μg/mL ampicillin. One mL was inoculated into 100 mL of LB broth+100 μg/mL ampicillin shaken at 180 rpm for 4 hours at 37° C. before being induced with 1 mM IPTG (final concentration) for 3 h under the same conditions. Cells were harvested at 4° C. and fixed in 0.5% (v/v) final concentration formalin (37%) on ice and incubated for 24 h at 4° C. Cells were pelleted and washed twice in PBS before being resuspended in PBS for IP injection. Control vaccine was prepared in an identical manner but with un-transformed BL21 cells.

### DNA Vaccine

The major sim gene type was inserted into a DNA vaccine vector using primers VF and VR which contained restriction sites for directional cloning into the DNA vector. The vector and PCR product were digested with BamHI and SalI. The construct was ligated and transformed into TOP10 cells (Invitrogen, Melbourne, Australia). Clones with the insert in the correct orientation (determined by sequencing) were grown overnight at 37° C. in 400 mL LB broth+50 μg/mL kanamycin, shaken at 150 rpm. Cells were harvested and plasmid was extracted using QIAGEN HiSpeed Plasmid Maxi Kit.

### Formalin Killed Bacterin

A positive control vaccine was prepared using strain QMA0155. The isolate was taken from stock and cultured overnight at 25° C. on blood agar. A single colony was emulsified in 1 mL vegetable peptone broth (VPB, Oxoid, Thebarton, Vic) and 500 μL was used to inoculate 50 mL VPB in a 250 mL Erlenmeyer flask. The culture was incubated with shaking (130 rpm) for 18 hours at 25° C. The resulting culture was immediately chilled to 4° C. on ice prior to inactivation with formalin (37%) at a concentration of 0.5 mL per 100 mL, to give a final formalin concentration of 0.2%. The culture was inactivated at 4° C. and checked for inactivation by culture of an aliquot on blood agar after 24 h and 48 h.

### Vaccination of Fish

Fish were starved for 24 hours prior to vaccination. Fish (80 per vaccine) were anaesthetised in clean aquarium water containing 0.01% MS222 and vaccinated either by intraperitoneal injection with 100 μL of vaccine preparation (Recombinant vaccine and controls) or by intramuscular injection with 5 μg DNA vaccine or vector control in 50 μL sterile PBS, just anterior of the tail. Fish were allowed to recover prior to returning to their tanks. Vaccine and control groups were maintained in separate tanks to allow development of immune response. Fish returned to feeding the day following vaccination and were maintained at 29° C. for 4 weeks prior to challenge. After 24 h and 48 h post-vaccine, 2 fish from the DNA vaccinated group were euthanased and approx 5 cubic millimetres of muscle removed from the injection site. RNA was isolated using a commercial kit (Qiagen) and DNA removed by digestion with DNase. RNA was reverse transcribed with superscript III reverse transcriptase, and a PCR was performed using primers directed against the emm gene to check for expression. Plasmid primers were used to check for plasmid DNA contamination of the RNA to verify that the emm gene was expressed.

### Preparation of the Challenge Inoculum

Streptococcus iniae strain QMA00155, isolated from a recirculating barramundi farm in New South Wales, Australia in 2006 was selected for this study. The isolate was cultured from stock (−80° C., 20% glycerol in vegetable peptone broth) on Columbia agar base containing 5% defibrinated sheep blood overnight at 25° C. Identity was verified by PCR of the lactate oxidase gene as previously described. For preparation of the challenge inoculum, a single colony was selected from the confirmed overnight agar culture and suspended in 1 ml sterile Tryptone soya broth (TSB, Oxoid, Thebarton, Vic). Aliquots (200 μl) were used to inoculate 20 mL TSB in 50 mL flasks and incubated for 18 h at 25° C. with gentle agitation (130 rpm). These late exponential phase cultures were washed in sterile PBS and resuspended to an optical density of 1.2 at 600 nm, previously estimated to be equivalent to approximately 5×10⁹ cfu mL⁻¹. This suspension was then diluted tenfold in PBS and used immediately for the challenge model.

### Injection Challenge Model

Fish were removed from their tanks using a hand net and anaesthetised in 0.01% MS222 in clean aquarium water. Each fish received 100 μL (5×10⁷ CFU) intraperitoneally by injection using a 1 mL tuberculin syringe fitted with a 25 G needle. Fish were marked according to their vaccine or control group (Table 4) with saturated Alcian blue in sterile PBS using a PanJet. Eight fish per vaccine or control group were distributed into 160 L plastic tanks connected to independent EHEIM pump filters in a separate quarantined aquarium room such that each vaccine/control group was represented in every tank. Mortalities or moribund fish were removed on first observation and head kidney sampled onto TSA. Overnight growth on TSA plates was subsequently sampled for direct-lysis PCR of the lox gene to confirm cause of death.

### Analysis of Results

Mortality data were analysed by Kaplan-Meyer survival analyses using Prism version 4 for Macintosh (GraphPad Ltd, California).

### Results

Twenty-six days into the trial, 18 hours prior to the proposed challenge, a major power failure knocked out the recirculating pumps and the air supply, resulting in the death of many of the fish by asphyxiation. Oxygen was applied to the tanks until power was restored. There were 8 survivors in group 1 (DNA vaccine), 18 survivors in group 2 (B21 recombinant control), 19 survivors in group 3 (BL21 simA recombinant vaccine) and 20 survivors in group 4 (NAV vector control). There were no survivors in group 5, the positive control formalin killed homologous bacterin. The challenge was continued on a reduced scale with the survivors after a period of recovery from stress and return to feeding.

### Vaccine Efficacy

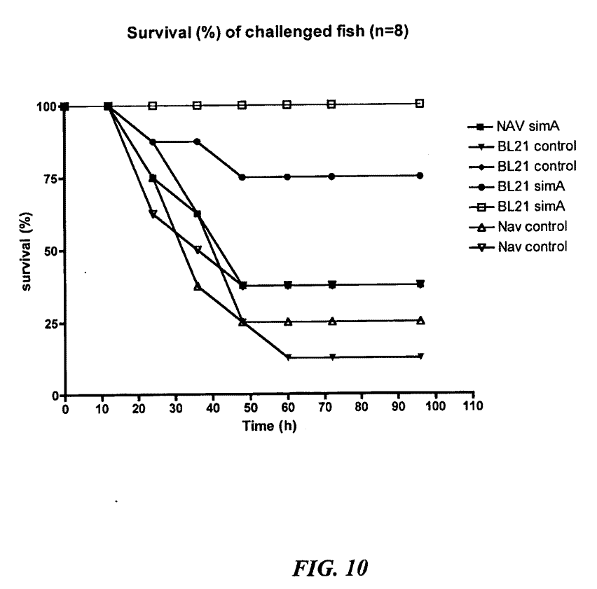

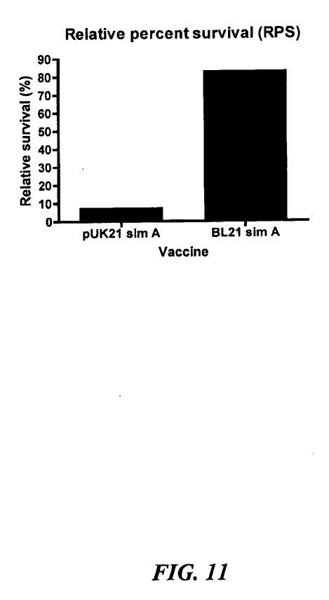

Eight fish from each of the surviving groups were challenged and marked and placed into a single tank. Where possible, the remaining survivors in each group were challenged, marked and distributed to a second tank to provide some replication. Thus the two tanks used contained 32 fish (8 from each of 4 groups) (Tank A) and 23 fish (8 from each of groups 2 and 4, and 7 from group 3) in tank B. All fish recovered from anaesthesia and appeared health 12 hours post challenge. First mortalities appeared 24 h post-challenge with red lesions along the ventral surface apparent in most cases. Moralities continued until 72 hours post-challenge. No further mortalities were recorded. Mortalities ranged from 0 to 75% and are shown in FIG. 10. Maximum mortality was recorded in the BL21 recombinant control group, whilst the lowest mortality was recorded in the BL21 simA recombinant vaccine groups (0% (0/7) and 25% (2/8) in the 2 replicates). Relative percent survival (RPS) was calculated for the DNA vaccine pUK21A2-simA at 7.76% whilst RPS for the recombinant vaccine expressed in E. coli was 83.3% (FIG. 11)

### Discussion

While many of the fish were lost the night prior to challenge, some fish were saved and a reduced experiment yielded data. Both the DNA vaccine and the recombinant protein vaccine demonstrated protection, although the recombinant protein vaccine was superior. The challenge model achieved 75% mortality in control groups, perhaps reflecting preparation of the inoculum into exponential phase in broth at 25° C., rather than growth on plates at 37° C. Virulence determinants, including capsule and M protein may be both temperature dependent and phase variable. As vaccinates and controls were challenged together in the same tank, there was high reproducibility between control groups, and strong performance of the recombinant vaccine, the low number of replicates was abrogated to some degree. The recombinant protein vaccine showed an RPS of 83.3%. Several DNA vaccinated fish showed expression 24 h and 48 h post vaccination (FIG. 4).

The invention has been described herein accordingly and is not to be construed as limited to any one aspect described herein. Various changes and modifications may be made to the aspects described and illustrated herein without departing from the broad spirit and scope of the invention.

All patent and scientific literature, computer programs and algorithms referred to herein are incorporated by reference in their entirety.

TABLE 1

Primers.

| | | |
|---|---|---|
| Primer | Sequence (5′-3′) | Identifier |

| | | |
|---|---|---|
| ALL MF | GGGGGGGGATCCATAAGGAGCATAAAAATGGCT | SEQ ID NO: 18 |
| ALL MR | GGGGGGGAATTCAGCTTAGTTTTCTTCTTTGCG | SEQ ID NO: 19 |
| SP6 | GCTATTTAGGTGACACTATAGAAT | SEQ ID NO: 20 |
| T7 | GTAATACGACTCACTATAGGG | SEQ ID NO: 21 |
| SIM F | AATTAATGAAGCTGGAGTGCTCT | SEQ ID NO: 22 |
| SIM WF | GGGAGGCTTCGCTGACATTTATTTCC | SEQ ID NO: 23 |
| SIM W3F | TACGGCCGTAAACGCAAAGAGGAAGAA | SEQ ID NO: 24 |
| SIM R | GCTTCCACAAGTTTTTCTTTGTCA | SEQ ID NO: 25 |
| SIM 2R | GTAGATAGGCTTTGATTTTCACTA | SEQ ID NO: 26 |
| SIM 2F | GATACGCTTCAAAGTTCTTACTAT | SEQ ID NO: 27 |
| SIM 3F | GCTGCTAAGATCAACATGCC | SEQ ID NO: 28 |
| SIM WR | ACCTATAATCAGGTTCTAAATTCGTGGC | SEQ ID NO: 29 |
| SIM W2R | GGAAGATGTGTCCATTGTTTGATGAGATG | SEQ ID NO: 30 |
| PRE SIM | TTGTTGGGTGGAAAAAAGATC | SEQ ID NO: 31 |
| POST | AAACTCAGGGACCAAAAAATTG | SEQ ID NO: 32 |
| SIM | | |
| SIM3F | GGCATGTTGATCTTAGCAGC | SEQ ID NO: 33 |
| RC | | |
| M141 F | CAAAATGATCACATCAGC | SEQ ID NO: 34 |
| ESIM | CACCATGGCTAAACAAATCAAAGC | SEQ ID NO: 35 |
| 20F | | |
| ESIM | TTCTTCCTCTTTGCGTTTACGG | SEQ ID NO: 36 |
| 1542R | | |

TABLE 2
S. iniae simA gene lengths and M protein identity and similarities to emm-like
proteins from other streptococci.

| | Length (nt, | | Identity | Similarity | | sim |
|---|---|---|---|---|---|---|
| Strain | aa) | BLAST-P result | (%)^{a} | (%)^{b} | Gene | sequevar |
| QMA0072 | 1569, 522 | S. dysgalactiae demA | 32 | 49 | simA | A2 |
| | | gene | | | | |
| QMA0076 | 1566, 521 | S. dysgalactiae demA | 32 | 50 | simA | A1 |
| | | gene | | | | |
| QMA0141 | 1740, 579 | S. dysgalactiae subsp. | 31 | 48 | simB | B1 |
| | | dysgalactiae M-like | | | | |
| | | protein | | | | |

^{a,b}% according to BLASTP analysis.

TABLE 3
Primers used for constructing recombinant
protein vaccine.

| Primer | Sequence (5′-3′) | |
|---|---|---|
| ESIM | CACCATGGCTAAACAAATCAAAGC | SEQ ID NO: 35 |
| 20F | | |
| ESIM | TTCTTCCTCTTTGCGTTTACGG | SEQ ID NO: 36 |
| 1542R | | |
| VF | AAGGATCCATATGGCTAAACAAATCAAAGC | SEQ ID NO: 37 |
| VR | TTGTCGACTTATTCTTCCTCTTTGCGTTTAC | SEQ ID NO: 38 |

TABLE 4
Vaccine groups and tank allocation at challenge

| Group | Vaccine | Mark | Tank A | Tank B |
|---|---|---|---|---|
| 1 | pUK21A2 simA (5 μg) | Throat | 8 | 0 |
| 2 | BL21 star | Pre-pectoral | 8 | 8 |
| 3 | BL21 star pET-simA | Post-pectoral | 8 | 7 |
| 4 | pUK21A2 (5 μg) | Anal | 8 | 8 |
| 5 | QMA0165 killed | n/a | 0 | 0 |

1. An isolated M protein, or M-like protein, of Streptococcus iniae.

2. The isolated M protein, or M-like protein, of claim 1, comprising an amino acid sequence selected from the group consisting of: RLTLEEKMEALRKVVT (SEQ ID NO: 1) and KMAEIQEEANKKIAA (SEQ ID NO:2).

3. The isolated M protein, or M-like protein, of claim 1, comprising an amino acid sequence according to SEQ ID NO: 3 or SEQ ID NO:4.

4. An isolated protein having reduced or lower fibrinogen binding compared to the isolated protein of claim 3, said isolated protein comprising an amino acid sequence of an S. iniae M protein, or M-like protein, but which does not comprise an amino acid sequence selected from the group consisting of: RLTLEEKMEALRKVVT (SEQ ID NO:1); KMAEIQEEANKKIAA (SEQ ID NO:2); and one or more of residues 190-220 of SEQ ID NO:3 or SEQ ID NO:4.

5. The isolated protein of claim 4, comprising an amino acid sequence selected from the group consisting of: SEQ ID NO: 5; SEQ ID NO:6 and SEQ ID NO:7.

6. An isolated protein comprising an amino acid sequence selected from the group consisting of: SEQ ID NO:1; SEQ ID NO:2; SEQ ID NO:3; SEQ ID NO:4; SEQ ID NO:5; SEQ ID NO:6; and SEQ ID NO:7.

7. (canceled)

8. An immunogenic fragment, comprising 20-200 contiguous amino acids of an amino acid sequence selected from the group consisting of: SEQ ID NO: 1; SEQ ID NO:2; SEQ ID NO:3; SEQ ID NO:4; SEQ ID NO:5; SEQ ID NO:6; and SEQ ID NO:7.

9. An immunogenic polypeptide, comprising an amino acid sequence at least 75% identical to an amino acid sequence selected from the group consisting of: SEQ ID NO:1; SEQ ID NO:2; SEQ ID NO:3; SEQ ID NO:4; SEQ ID NO:5; SEQ ID NO:6; and SEQ ID NO:7.

10. An antibody or antibody fragment which binds an isolated M protein, or M-like protein, of Streptococcus iniae, or a fragment, variant or derivative of an isolated protein comprising an amino acid sequence selected from the group consisting of: SEQ ID NO:1, SEQ ID NO:2, SEQ ID NO:3, SEQ ID NO:4, SEQ ID NO:5, SEQ ID NO:6, and SEQ ID NO:7.

11. An isolated nucleic acid encoding an isolated M protein, or M-like protein, of Streptococcus iniae, or a fragment, variant or derivative of an isolated protein comprising an amino acid sequence selected from the group consisting of: SEQ ID NO:1, SEQ ID NO:2, SEQ ID NO:3, SEQ ID NO:4, SEQ ID NO:5, SEQ ID NO:6, and SEQ ID NO:7.

12. The isolated nucleic acid of claim 11, comprising a nucleotide sequence selected from the group consisting of: SEQ ID NO:8; SEQ ID NO:9; SEQ ID NO:10; SEQ ID NO:11; and SEQ ID NO:39.

13. A genetic construct comprising the isolated nucleic acid of claim 11.

14. A genetic construct according to claim 13 which is suitable for bacterial expression of a recombinant M protein of S. iniae.

15. A genetic construct according to claim 13 which is suitable for DNA vaccination of a fish.

16. A host cell comprising the genetic construct of claim 13.

17. A host cell according to claim 16, which is a bacterial cell.

18. A host cell according to claim 16, which is a fish cell.

19. A composition comprising at least one isolated M protein, or M-like protein, of Streptococcus iniae according to claim 1; and an acceptable carrier, diluent, or excipient.

20. The composition of claim 19, in the form of a vaccine capable of eliciting a protective immune response against S. iniae in an animal.

21. (canceled)

22. (canceled)

23. A method of treating an S. iniae infection of an animal, said method including the step of administering a composition according to claim 19 to said animal to thereby treat said S. iniae infection.

24. The method of claim 23 wherein the animal is a fish.

25. The method of claim 24 wherein the animal is a human.

26. A method of immunizing an animal against S. iniae infection, said method including the step of administering a composition according to claim 20 to said animal to thereby immunize said animal against S. iniae infection.

27. The method of claim 26 wherein the animal is a fish.

28. The method of claim 26 wherein the animal is a human.

29-31. (canceled)

32. A method of determining whether an animal is, or has been, exposed to S. iniae bacteria or components thereof, said method comprising determining whether a biological sample obtained from said animal comprises an isolated protein according to claim 1, a fragment according to claim 8, or a polypeptide according to claim 9, a presence of which indicates that said animal is, or has been, exposed to S. iniae bacteria or components thereof.

33. A method of determining whether an animal is, or has been, exposed to S. iniae bacteria or components thereof, said method comprising determining whether a biological sample obtained from said animal comprises an isolated nucleic acid according to claim 11, a presence of which indicates that said animal is, or has been, exposed to S. iniae bacteria or components thereof.

34. A method of determining whether an animal is, or has been, exposed to S. iniae bacteria or components thereof, said method including the step of determining whether a biological sample obtained from said animal comprises an antibody or antibody fragment according to claim 10, a presence of which indicates that said animal is, or has been, exposed to S. iniae bacteria or components thereof.

35. (canceled)

36. (canceled)

37. A diagnostic kit or composition for detecting an S. iniae bacterium, molecular component(s) thereof and/or an antibody thereto, in a biological sample obtained from an animal, said kit or composition comprising one or more detection agents selected from the group consisting of: an isolated protein according to claim 1, a fragment according to claim 8, a polypeptide according to claim 9, an antibody or antibody fragment according to claim 10 and an isolated nucleic acid according to claim 11 in a biological sample obtained from an animal.

38. The diagnostic kit or composition of claim 37, further comprising one or more detection reagents.


SEQUENCE LISTING (39 sequences)

SEQ ID NO: 1 - protein, 16 aa, Streptococcus iniae
RLTLEEKMEALRKVVT

SEQ ID NO: 2 - protein, 15 aa, Streptococcus iniae
KMAEIQEEANKKIAA

SEQ ID NO: 3 - protein, 522 aa, Streptococcus iniae
MAKQIKARKHALRKMITSAVLAGTAITTIGGAMGSVTTVKADSDRLTLEEKMEALRKVVTREVLIGYANNNPRFGFWMSLQQLEKEIDKTQRLTWENKRMEETLKSKVERINEAGVLLSKKQKDLNEAEAKITDLNSKQTDLTNQKEQVEKELKDTKDKLKDSIANASRIAEHEAIRSAGLENQVKSLRDVTKSLVSTIINTLTKESATIKAKMAEIQEEANKKIAAVQTELENIDSENQSLSTANEQLKADLEQAARELDTLQSSYYTVENEKAELQKQLAEKDAKIAELEANNTELTATVADLTKALEAAKKEAEEKPALKAKVAELEKALAEAKGLGTKVAELEKDLEKAQAEAKDLETKLAETKAELEKVQAEKAELEATIEKMKKEHAEELDKLNALLADKEKLVEALNKEIEALKKDFDEKSNHSAQEKAKFQEELERLKKELAAKINMPMGNTKGMANAAGNAQTPANNGQNNAVKNQLPSTGDKAGNPFFTASAIAVMVGAGTLAYGRKRKEEE

SEQ ID NO: 4 - protein, 521 aa, Streptococcus iniae
MAKQIKARKHALRKMITSAVLAGTAITTIGGAMGSVTTVKADSDRLTLEEKMEALRKVVTREVLIGYANNNPRFGFWMSLQQLEKEIDKTQRLTWENKRMEETLKSKVERINEAGVLLSKKQKDLNEAEAKITDLNSKQTDLTNQKEQVEKELKDTKDKLKDSIANASRIAEHEAIRSAGLENQVKSLRDVTKSLVSTINTLTKESATIKAKMAEIQEEANKKIAAVQTELENIDSENQSLSTANEQLKADLEQAARELDTLQSSYYTVENEKAELQKQLAEKDAKIAELEANNTELTATVADLTKALEAAKKEAEEKPALKAKVAELEKALAEAKGLGTKVAELEKDLEKAQAEAKDLETKLAETKAELEKVQAEKAELEATIEKMKKEHAEELDKLNALLADKEKLVEALNKEIEALKKDFDEKSNHSAQEKAKFQEELERLKKELAAKINMPMGNTKGMANAAGNAQTPANNGQNNAVKNQLPSTGDKAGNPFFTASAIAVMVGAGTLAYGRKRKEEE

SEQ ID NO: 5 - protein, 579 aa, Streptococcus iniae
MAKQIKARKHALRKMITSAVLAGTAITTIGGAMGSVKSVYALPKIEEEVSTGLNKIIAERRINNKYSIQNNDEETVEKIKDLLRGANKETLERLSDGFNPYSPQYLDSMLTYFEHKIGPLRNGKDSVSLRTLLVEVFREKFDESIVFKQLANGAETALRKKEEEFKLASEKFSKKKNEMEASLNKLEEKGKELSDKLSMADKKLASKEEELKVTKENLQSMIESASKMAKHEAIRSAGLEDQVKSLRETTKSLVSTIQDLTGRSEAIKAQMADIQAQAAQKIAAVEAELENIGSENRTLEAINEEIQNELASTKRDLDALESSLYKVENEKTALMAQLSEKEAKIAELEAGNAELTTSVADLTKALEAAKKEVEENPALKAKVAELEKALAEAKGLEAKVAELEKALEKAQAEAKELEAKLAETKAELEKVQAEKAELEATIETMKKEHAEELDKLNALLADKEKLNEELNKMIADLKKELENQANLSNEEKAQLKAELEKLKQELAAKINMPMGNTKGMANGAGNAQTQANNGQNNTVKNQLPSTGDKAGNPFFTASAIAVMVGAGTLAYGRKRKEEE

SEQ ID NO: 6 - protein, 202 aa, Streptococcus iniae
MAKQIKARKHALRKMITSAVLAGTAITTIGGAMGSVTTVKADSDRLTLEEKMEALRKVVTREVLIGYANNNPRFGFWMSLQQLEKEIDKTQRLTWENKRMEETLKSKVERINEAGVLLSKKQKDLNEAEAKITDLNSKQTDLTNQKEQVEKELKDTKDKLKDSIANASRIAEHEAIRSAGLENQVKSLRDVTKSLVSTEIIT

SEQ ID NO: 7 - protein, 309 aa, Streptococcus iniae
MAEIQEEANKKIAAVQTELENIDSENQSLSTANEQLKADLEQAARELDTLQSSYYTVENEKAELQKQLAEKDAKIAELEANNTELTATVADLTKALEAAKKEAEEKPALKAKVAELEKALAEAKGLGTKVAELEKDLEKAQAEAKDLETKLAETKAELEKVQAEKAELEATIEKMKKEHAEELDKLNALLADKEKLVEALNKEIEALKKDFDEKSNHSAQEKAKFQEELERLKKELAAKINMPMGNTKGMANAAGNAQTPANNGQNNAVKNQLPSTGDKAGNPFFTASAIAVMVGAGTLAYGRKRKEEE

SEQ ID NO: 8 - DNA, 1569 bp, Streptococcus iniae
atggctaaacaaatcaaagcccgtaaacatgccttgcgcaaaatgatcacatcagcagtccttgccggaacagcaatcacaaccatcggtggtgcaatgggaagcgtcacaacagtaaaagcggatagtgatagattaaccttggaagaaaaaatggaagcgttaagaaaggtagttactagagaagtattaataggttatgctaataataatccaagatttggtttttggatgtcgttacaacaattagaaaaagaaattgataaaacccaacgtttaacttgggaaaataaaagaatggaagagactctaaaaagtaaagttgaaagaattaatgaagctggagtgctcttatcaaagaaacaaaaagatttaaatgaagcagaagcaaaaattacagatcttaattcaaaacaaacggatttaacaaatcaaaaagaacaagtagaaaaagaattaaaagatactaaagataaacttaaagattcgattgctaatgcttcaaggattgctgagcatgaagcaattcgctcagcaggtcttgaaaatcaagtgaaatcattacgtgatgtaacaaagtcacttgtttcaactatcatcaatactttgactaaagagtcagcaacaattaaagctaaaatggctgaaattcaagaagaggctaataaaaaaattgcagcagtacaaacagaattagaaaatattgatagtgaaaatcaaagcctatctacagctaacgagcaattaaaagcagatttggaacaagctgcacgagaattagatacgcttcaaagttcttactatacagtagaaaatgaaaaggctgaacttcaaaaacaattggcagaaaaagatgctaagattgcggaacttgaagcaaacaacacagaattaacagcaactgttgctgatttgactaaagcattagaagcggctaagaaagaagcagaagaaaaacctgctcttaaagctaaagttgcagaattagaaaaagctcttgcagaagctaaaggtttagggactaaagttgcagaacttgaaaaagaccttgagaaagcacaagcagaagctaaagaccttgaaactaaactagcagaaacaaaagctgaattggaaaaagttcaagctgaaaaagcagaacttgaagcaactattgaaaaaatgaagaaagagcatgctgaagagcttgacaaactaaatgctcttcttgctgacaaagaaaaacttgtggaagctttgaacaaagaaatcgaagcgcttaagaaagactttgacgaaaaatcaaaccattctgcacaagaaaaagctaagttccaagaagaactggaacgcctcaaaaaagaattggctgctaagatcaacatgccaatgggcaacaccaaaggcatggcaaacgctgctggaaacgcgcaaactccagctaacaacggtcaaaacaacgctgttaagaaccaattgccatcaacaggtgataaagcaggtaacccattcttcacagcaagtgctatcgcggttatggttggagctggtactctagcatacggccgtaaacgcaaagaggaagaataa

SEQ ID NO: 9 - DNA, 1566 bp, Streptococcus iniae
atggctaaacaaatcaaagcccgtaaacatgccttgcgcaaaatgatcacatcagcagtccttgccggaacagcaatcacaaccatcggtggtgcaatgggaagcgtcacaacagtaaaagcggatagtgatagattaaccttggaagaaaaaatggaagcgttaagaaaggtagttactagagaagtattaataggttatgctaataataatccaagatttggtttttggatgtcgttacaacaattagaaaaagaaattgataaaacccaacgtttaacttgggaaaataaaagaatggaagagactctaaaaagtaaagttgaaagaattaatgaagctggagtgctcttatcaaagaaacaaaaagatttaaatgaagcagaagcaaaaattacagatcttaattcaaaacaaacggatttaacaaatcaaaaagaacaagtagaaaaagaattaaaagatactaaagataaacttaaagattcgattgctaatgcttcaaggattgctgagcatgaagcaattcgctcagcaggtcttgaaaatcaagtgaaatcattacgtgatgtaacaaagtcacttgtttcaactatcaatactttgactaaagagtcagcaacaattaaagctaaaatggctgaaattcaagaagaggctaataaaaaaattgcagcagtacaaacagaattagaaaatattgatagtgaaaatcaaagcctatctacagctaacgagcaattaaaagcagatttggaacaagctgcacgagaattagatacgcttcaaagttcttactatacagtagaaaatgaaaaggctgaacttcaaaaacaattggcagaaaaagatgctaagattgcggaacttgaagcaaacaacacagaattaacagcaactgttgctgatttgactaaagcattagaagcggctaagaaagaagcagaagaaaaacctgctcttaaagctaaagttgcagaattagaaaaagctcttgcagaagctaaaggtttagggactaaagttgcagaacttgaaaaagaccttgagaaagcacaagcagaagctaaagaccttgaaactaaactagcagaaacaaaagctgaattggaaaaagttcaagctgaaaaagcagaacttgaagcaactattgaaaaaatgaagaaagagcatgctgaagagcttgacaaactaaatgctcttcttgctgacaaagaaaaacttgtggaagctttgaacaaagaaatcgaagcgcttaagaaagactttgacgaaaaatcaaaccattctgcacaagaaaaagctaagttccaagaagaactggaacgcctcaaaaaagaattggctgctaagatcaacatgccaatgggcaacaccaaaggcatggcaaacgctgctggaaacgcgcaaactccagctaacaacggtcaaaacaacgctgttaagaaccaattgccatcaacaggtgataaagcaggtaacccattcttcacagcaagtgctatcgcggttatggttggagctggtactctagcatacggccgtaaacgcaaagaggaagaataa

SEQ ID NO: 10 - DNA, 1740 bp, Streptococcus iniae
atggctaaacaaatcaaagcccgtaaacatgccttgcgcaaaatgatcacatcagcagtccttgccggaacagcaatcacaaccatcggtggcgcaatgggaagcgtaaaatctgtttacgcactaccgaaaattgaagaagaagtaagtactggattaaataaaataattgctgaaagaagaataaataataaatattctatacaaaataatgatgaagaaacggtagaaaaaattaaagatttattgagaggggctaataaagaaacactagagcgtttatcagatggatttaatccttatagtcctcagtatcttgatagcatgcttacttattttgaacataaaataggcccattacgaaacggaaaagattcagtctctttgagaacattattagttgaggtatttagagaaaaatttgacgaaagtattgtttttaaacaactagcaaatggggcagaaactgctttacgcaaaaaggaagaagaatttaaacttgcttcagaaaagttttctaagaaaaaaaacgagatggaagcttctttaaataaattggaagaaaaaggaaaagagcttagcgataaacttagtatggctgataaaaagcttgcttctaaagaagaagaattaaaggttacaaaagaaaacttgcagtctatgattgaaagtgcttcaaaaatggctaaacatgaagcaattcgctcagcaggtcttgaagatcaagtgaaatcactacgtgaaacaacaaaatcacttgtttcaacaatccaagatttaacaggacgttcagaagcaattaaagcccaaatggctgatattcaagcacaagcagctcaaaaaattgctgcagtagaagctgagttggaaaatattggtagtgaaaatagaacattagaagctattaatgaagaaatccaaaatgaattagcttctacaaagcgtgacctagatgctttagaaagctctctttacaaagttgaaaatgaaaaaacagctttaatggctcagttatctgaaaaagaagctaagattgctgaacttgaagctggtaatgcagaattaacaacttctgttgctgatttgacaaaagctttagaagcggctaagaaagaagtagaagaaaatcctgctcttaaagctaaagttgcagaattggaaaaagctcttgcagaagctaaaggcttagaggctaaagttgcagaacttgaaaaagctcttgagaaagcacaagcagaagctaaagaactagaagctaaactagcggaaacaaaagctgaattggaaaaagttcaagcggaaaaagcagaacttgaagcaactattgagacaatgaagaaagagcatgctgaagagcttgacaaactaaatgctcttcttgctgacaaagaaaaactcaacgaagaacttaataaaatgattgcagaccttaagaaagaacttgaaaaccaagcaaacctttcaaatgaggaaaaagcacaacttaaagcagaacttgaaaaacttaaacaagaattggctgctaagatcaacatgccaatgggcaacaccaaaggtatggcaaacggtgctggaaacgcgcaaacacaagcaaataatggtcaaaacaacactgttaagaaccaattgccatcaacaggtgataaagcaggtaacccattcttcaccgcaagtgctatcgcggttatggttggagctggtactctagcgtacggccgtaaacgcaaagaggaagaataa

SEQ ID NO: 11 - DNA, 1606 bp, Streptococcus iniae
atggctaaacaaatcaaagcccgtaaacatgccttgcgcaaaatgatcacatcagcagtccttgccggaacagcaatcacaaccatcggtggtgcaatgggaagcgtcacaacagtaaaagcggatagtgatagattaaccttggaagaaaaaatggaagcgttaagaaaggtagttactagagaagtattaataggttatgctaataataatccaagatttggtttttggatgtcgttacaacaattagaaaaagaaattgataaaacccaacgtttaacttgggaaaataaaagaatggaagagactctaaaaagtaaagttgaaagaattaatgaagctggagtgctcttatcaaagaaacaaaaagatttaaatgaagcagaagcaaaaattacagatcttaattcaaaacaaacggatttaacaaatcaaaaagaacaagtagaaaaagaattaaaagatactaaagataaacttaaagattcgattgctaatgcttcaaggattgctgagcatgaagcaattcgctcagcaggtcttgaaaatcaagtgaaatcattacgtgatgtaacaaagtcacttgtttcaactgaaatcattacgtgatgtaacaaagtcacttgtttcaactatcaatactttgactaaagagtcagcaacaattaaagctaaaatggctgaaattcaagaagaggctaataaaaaaattgcagcagtacaaacagaattagaaaatattgatagtgaaaatcaaagcctatctacagctaacgagcaattaaaagcagatttggaacaagctgcacgagaattagatacgcttcaaagttcttactatacagtagaaaatgaaaaggctgaacttcaaaaacaattggcagaaaaagatgctaagattgcggaacttgaagcaaacaacacagaattaacagcaactgttgctgatttgactaaagcattagaagcggctaagaaagaagcagaagaaaaacctgctcttaaagctaaagttgcagaattagaaaaagctcttgcagaagctaaaggtttagggactaaagttgcagaacttgaaaaagaccttgagaaagcacaagcagaagctaaagaccttgaaactaaactagcagaaacaaaagctgaattggaaaaagttcaagctgaaaaagcagaacttgaagcaactattgaaaaaatgaagaaagagcatgctgaagagcttgacaaactaaatgctcttcttgctgacaaagaaaaacttgtggaagctttgaacaaagaaatcgaagcgcttaagaaagactttgacgaaaaatcaaaccattctgcacaagaaaaagctaagttccaagaagaactggaacgcctcaaaaaagaattggctgctaagatcaacatgccaatgggcaacaccaaaggcatggcaaacgctgctggaaacgcgcaaactccagctaacaacggtcaaaacaacgctgttaagaaccaattgccatcaacaggtgataaagcaggtaacccattcttcacagcaagtgctatcgcggttatggttggagctggtactctagcatacggccgtaaacgcaaagaggaagaataa

SEQ ID NO: 12 - protein, 548 aa, Streptococcus dysgalactiae
MPKTIKARKHALRKAVTAAVLMGTAVTTVGGALGATTTVKANDDIYDIAASRIVNDKLSDTYLKKLLDIHSGSGFDPSLAPVLLFKAKEEAMNRIGRLRNEIKGLEKERDLFIDVANKTDEARAATQEKLNNKVSELSEKEKALKATLAETKKLSTDLSSAQKERDTAFKVSKDLADKLAQSEKAKTNALNVSKDLADKLADSEKGKANALNVSKDLADKLAESEKAKANALNVSKDLADKLAAAEASRDKAFAVSTDLANQLAAKTAEAEDLLNTVDSFGRLVESAKREMQEKLAEIDRLKAEKAQSDAALEAANATIAELQAEVEKVKSDLANANQLVEAGKRDLSDLAKAKADADAALVASEAKVAELKKAKAESEAKVAKLESAVTAAKEEVAKVKAELETVTKDLEAVKAEKANLEAQIADLKKAHAEKIAELEATIKRLEEELAAKVKEFEALENTSKEEKANFQKEIDRLKAELAAKIKDITKSTKVGSSATTGMASSSVASNKVQLPSTGEATNAFFTAAALTIMAGAGVMAVAGKRKED

SEQ ID NO: 13 - protein, 6 aa, Artificial sequence
  Primer-encoded C terminal sequence
KRKEEN

SEQ ID NO: 14 - protein, 6 aa, Streptococcus iniae
KRKEEE

SEQ ID NO: 15 - DNA, 10 bp, Streptococcus iniae
aaataaggag

SEQ ID NO: 16 - protein, 5 aa, Streptococus iniae
LPSTG

SEQ ID NO: 17 - protein, 10 aa, Streptococcus iniae
HEAIRSAGLE

SEQ ID NO: 18 - DNA, 33 bp, Artificial sequence
  Primer
ggggggggatccataaggagcataaaaatggct

SEQ ID NO: 19 - DNA, 33 bp, Artificial sequence
  Primer
gggggggaattcagcttagttttcttctttgcg

SEQ ID NO: 20 - DNA, 24 bp, Artificial sequence
  Primer
gctatttaggtgacactatagaat

SEQ ID NO: 21 - DNA, 21 bp, Artificial sequence
  Primer
gtaatacgactcactataggg

SEQ ID NO: 22 - DNA, 23 bp, Artificial sequence
  Primer
aattaatgaagctggagtgctct

SEQ ID NO: 23 - DNA, 26 bp, Artificial sequence
  Primer
gggaggcttcgctgacatttatttcc

SEQ ID NO: 24 - DNA, 27 bp, Artificial sequence
  Primer
tacggccgtaaacgcaaagaggaagaa

SEQ ID NO: 25 - DNA, 24 bp, Artificial sequence
  Primer
gcttccacaagtttttctttgtca

SEQ ID NO: 26 - DNA, 24 bp, Artificial sequence
  Primer
gtagataggctttgattttcacta

SEQ ID NO: 27 - DNA, 24 bp, Artificial sequence
  Primer
gatacgcttcaaagttcttactat

SEQ ID NO: 28 - DNA, 20 bp, Artificial sequence
  Primer
gctgctaagatcaacatgcc

SEQ ID NO: 29 - DNA, 28 bp, Artificial sequence
  Primer
acctataatcaggttctaaattcgtggc

SEQ ID NO: 30 - DNA, 29 bp, Artificial sequence
  Primer
ggaagatgtgtccattgtttgatgagatg

SEQ ID NO: 31 - DNA, 21 bp, Artificial sequence
  Primer
ttgttgggtggaaaaaagatc

SEQ ID NO: 32 - DNA, 22 bp, Artificial sequence
  Primer
aaactcagggaccaaaaaattg

SEQ ID NO: 33 - DNA, 20 bp, Artificial sequence
  Primer
ggcatgttgatcttagcagc

SEQ ID NO: 34 - DNA, 18 bp, Artificial sequence
  Primer
caaaatgatcacatcagc

SEQ ID NO: 35 - DNA, 24 bp, Artificial sequence
  Primer
caccatggctaaacaaatcaaagc

SEQ ID NO: 36 - DNA, 22 bp, Artificial sequence
  Primer
ttcttcctctttgcgtttacgg

SEQ ID NO: 37 - DNA, 30 bp, Artificial sequence
  Primer
aaggatccatatggctaaacaaatcaaagc

SEQ ID NO: 38 - DNA, 31 bp, Artificial sequence
  Primer
ttgtcgacttattcttcctctttgcgtttac

SEQ ID NO: 39 - DNA, 1860 bp, Streptococcus iniae
ttaaaattgctcataaaaatgtaactattttaattataactattataacatttaataaaaggttataagctaaaaataccattaaaaaactgggctttattctcatttaaaactcatatatactaattttatagattgacccaacttgccctcaaagagcagcaagattgcaatcataatattaggaagaaaataaggagaaccctaaacatggctaaacaaatcaaagcccgtaaacatgccttgcgcaaaatgatcacatcagcagtccttgccggaacagcaatcacaaccatcggtggtgcaatgggaagcgtcacaacagtaaaagcggatagtgatagattaaccttggaagaaaaaatggaagcgttaagaaaggtagttactagagaagtattaataggttatgctaataataatccaagatttggtttttggatgtcgttacaacaattagaaaaagaaattgataaaacccaacgtttaacttgggaaaataaaagaatggaagagactctaaaaagtaaagttgaaagaattaatgaagctggagtgctcttatcaaagaaacaaaaagatttaaatgaagcagaagcaaaaattacagatcttaattcaaaacaaacggatttaacaaatcaaaaagaacaagtagaaaaagaattaaaagatactaaagataaacttaaagattcgattgctaatgcttcaaggattgctgagcatgaagcaattcgctcagcaggtcttgaaaatcaagtgaaatcattacgtgatgtaacaaagtcacttgtttcaactatcaatactttgactaaagagtcagcaacaattaaagctaaaatggctgaaattcaagaagaggctaataaaaaaattgcagcagtacaaacagaattagaaaatattgatagtgaaaatcaaagcctatctacagctaacgagcaattaaaagcagatttggaacaagctgcacgagaattagatacgcttcaaagttcttactatacagtagaaaatgaaaaggctgaacttcaaaaacaattggcagaaaaagatgctaagattgcggaacttgaagcaaacaacacagaattaacagcaactgttgctgatttgactaaagcattagaagcggctaagaaagaagcagaagaaaaacctgctcttaaagctaaagttgcagaattagaaaaagctcttgcagaagctaaaggtttagggactaaagttgcagaacttgaaaaagaccttgagaaagcacaagcagaagctaaagaccttgaaactaaactagcagaaacaaaagctgaattggaaaaagttcaagctgaaaaagcagaacttgaagcaactattgaaaaaatgaagaaagagcatgctgaagagcttgacaaactaaatgctcttcttgctgacaaagaaaaacttgtggaagctttgaacaaagaaatcgaagcgcttaagaaagactttgacgaaaaatcaaaccattctgcacaagaaaaagctaagttccaagaagaactggaacgcctcaaaaaagaattggctgctaagatcaacatgccaatgggcaacaccaaaggcatggcaaacgctgctggaaacgcgcaaactccagctaacaacggtcaaaacaacgctgttaagaaccaattgccatcaacaggtgataaagcaggtaacccattcttcacagcaagtgctatcgcggttatggttggagctggtactctagcatacggccgtaaacgcaaagaggaagaataagctttcccttgcaaccttttcatagacacaccccctataagtccactggacttatagggggttttgacattcaaaactcctact